(19) United States
(12) Patent Application Publication
(10) Pub. No.: US 2026/0027260 A1
(43) Pub. Date: Jan. 29, 2026

(54) TISSUE REPAIR SYSTEMS AND RELATED METHODS

(71) Applicant: Miach Orthopaedics, Inc., Westborough, MA (US)

(72) Inventors: Stephen WOHLERT, Flemington, NJ (US); Rita PAPARAZZO, Hopkinton, MA (US)

(73) Assignee: Miach Orthopaedics, Inc., Westborough, MA (US)

(21) Appl. No.: 19/280,945

(22) Filed: Jul. 25, 2025

Related U.S. Application Data
(60) Provisional application No. 63/676,038, filed on Jul. 26, 2024.
(60) Provisional application No. 63/740,568, filed on Dec. 31, 2024.

(51) Int. Cl. A61L 27/24; A61L 27/36

(52) U.S. Cl. CPC A61L 27/24; A61L 27/3616; A61L 27/3654; A61L 2430/06

(57) ABSTRACT

A tissue repair system is disclosed, comprising a collagen implant designed for repair of tissues, including meniscal tears and rotator cuffs, formed from self-assembled collagen fibers without cross-linking agents. The implant is absorbent and has structural integrity for suture securement. It is delivered through a specialized device, with sutures for securement. A delivery device, suture passer, and syringe are integral components of the system. The method involves accessing the tissue site, securing sutures, positioning and securing the implant, and injecting blood for tissue repair. The system and method facilitate tissue regeneration and repair in a minimally invasive manner, allowing for the growth of healthy tissue at the injury site, particularly for anterior cruciate ligament (ACL) and meniscus repair.

### CROSS-REFERENCE TO RELATED APPLICATIONS

The present application claims the benefit of and priority to U.S. Provisional Patent Application No. 63/740,568, filed on Dec. 31, 2024, and U.S. Provisional Patent Application. No. 63/676,038, filed on Jul. 26, 2024, the entire contents of which are incorporated by reference into the present application.

### TECHNICAL FIELD

The present disclosure relates to tissue repair systems and related methods, and particularly for tissue repair systems for soft tissues.

### BACKGROUND

Recent developments in scaffolds for tissue repair show promise in terms of improved health outcomes, faster healing, and few long-term adverse effects. Developing scaffolds and the ability to inject repair material directly to the site of the tissue defect or tear can improve healing.

### SUMMARY

An embodiment of the present disclosure relates to a tissue repair system for a rupture tear of a tissue (e.g., a meniscus, rotator cuff, etc.). The tissue repair system may include a collagen implant having a lower surface, an upper surface opposite the lower surface, and a side wall that extends from the lower surface to the upper surface. In embodiments, the side wall defines an outer perimeter that is shaped to fit between a femur and a tibia and along the tear of the meniscus when positioned adjacent to the meniscus. The collagen implant may have a self-assembly of collagen fibers and in various embodiments is substantially free of cross-linking agents. The collagen implant may be configured for securement to a first and second portion of a meniscus in order to facilitate growth of meniscal tissue at or near the tear.

In another embodiment of the disclosure, is a tissue repair system for a tear of a tissue (e.g., a meniscus, rotator cuff, etc.) comprising a collagen implant having a lower surface, an upper surface opposite the lower surface, and a side wall that extends from the lower surface to the upper surface. The side wall defines an outer perimeter that is shaped to fit between a femur and a tibia and along the tear of the meniscus when positioned adjacent to the meniscus. The collagen implant may be substantially free of cross-linking agents. In embodiments, the collagen implant is configured for securement to a first and second portion of a meniscus in order to facilitate growth of meniscal tissue at or near the tear. One or more suture assemblies may be configured to pass through a first portion of the meniscus and a second portion of the meniscus to secure the collagen implant in place on or along the tear of the meniscus. The system may further include a delivery device configured to carry the collagen implant for delivery at or near the tear of the meniscus. The delivery device may have an elongated body, a proximal end, a distal end opposite the proximal end, and a channel that extends from the distal end toward the proximal end, the channel defining a cross-sectional dimension that is shaped and sized to carry the collagen implant. In various embodiments, the delivery device includes a moveable rod in the channel and is configured to eject the collagen implant from the distal end of the delivery device into the tissue.

Further described herein according to various embodiments is a method for delivering a collagen implant to a ruptured or torn meniscus and facilitating growth of meniscal tissue. The method may include forming a portal in a knee joint to access a meniscus having a rupture or tear, and inserting a delivery device into the portal so that a distal end of the delivery device is positioned close to the meniscus. In embodiments, the method includes causing a collagen implant to exit the distal end of the delivery device in order to position it on or near the rupture or tear of the meniscus. The method may further include securing, with one or more suture assemblies, the collagen implant in place at or near the rupture or tear of the meniscus. The collagen implant may be a self-assembly of collagen fibers. In embodiments, the collagen implant is substantially free of cross-linking agents, and is configured to facilitate growth of meniscal tissue at or near the tear.

In further embodiments, described herein is a method for delivering a collagen implant to torn or ruptured anterior cruciate ligament (ACL), comprising: positioning a bone fixation device on a femur, wherein one or more suture assemblies are attached to the bone fixation device and securing the one or more suture assemblies to an ACL with a rupture or tear. In embodiments, the method includes threading a collagen implant along or on the one or more suture assemblies. The method may further include injecting a volume of blood into the collagen implant so that the blood is positioned at or adjacent the rupture or tear of the ACL and a rupture or tear of a meniscus. In embodiments, the method includes observing growth of the ACL and meniscal tissue at or near the rupture or tear of the ACL.

Further described herein according to embodiments is a method for positioning a collagen implant at or near a ruptured or torn ACL and observing growth of the ACL and meniscus tissue. The method may include positioning a bone fixation device on a femur. One or more suture assemblies may be attached to the bone fixation device. The method may further include securing the one or more suture assemblies to the ACL with a rupture or tear. In embodiments, the method includes injecting a volume of blood into a collagen implant and threading the collagen implant along or on the one or more suture assemblies so that the blood is positioned at or adjacent the rupture or tear of the ACL and a rupture or tear of a meniscus. The method may further include observing growth of the ACL and meniscal tissue at or near the rupture or tear of the ACL.

Another embodiment of the disclosure is a tissue repair system for a tear of a rotator cuff. The tissue repair system may include a collagen implant having a lower surface, an upper surface opposite the lower surface, and a side wall that extends from the lower surface to the upper surface, the side wall defining an outer perimeter that is shaped to fit in a shoulder joint along the tear on the rotator cuff, the collagen implant including a self-assembly of collagen fibers and being substantially free of cross-linking agents, wherein the collagen implant is configured for securement to a first and second portion of the tissue on the rotator cuff in order to facilitate growth of the tissue.

Further described herein according to various embodiments is a method for positioning a collagen implant at or near a ruptured or torn rotator cuff. The method may include attaching a first fixation device to a humerus and attaching a second fixation device to a rotator cuff. In embodiments, the method includes connecting one or more suture assemblies to the first and second fixation devices. The method may include placing a collagen implant injected with blood along or the one or more suture assemblies and positioning the collagen implant between ruptured or torn ends of the rotator cuff.

### BRIEF DESCRIPTION OF THE DRAWINGS

The foregoing summary, as well as the following detailed description of illustrative embodiments of the present application, will be better understood when read in conjunction with the appended drawings. For the purposes of illustrating the present application, there is shown in the drawings illustrative embodiments of the disclosure. It should be understood, however, that the application is not limited to the precise arrangements and instrumentalities shown:

FIG. 1A is a front view schematic illustration of a knee-joint;

FIG. 1B is a top view illustration of the knee joint shown in FIG. 1A with the femur removed;

FIG. 1C is a front view illustration of a shoulder joint with a partial rotator cuff tear;

FIG. 1D is a front view illustration of a shoulder joint with a complete rotator cuff tear;

FIG. 2 is a diagrammatic representation of the tissue repair system according to an embodiment of the present disclosure;

FIG. 3 is a diagrammatic representation of the implant shown in FIG. 2;

FIG. 4 is a diagrammatic representation of the implant shown in FIGS. 2-3;

FIG. 5 is a schematic depicting the tissue repair system being utilized for ligament repair in the ACL and meniscus repair;

FIG. 6 is a schematic depicting the implant shown in FIGS. 2-5 being inserted into a repair site;

FIG. 7 is a schematic depicting the tissue repair system shown in FIGS. 2-5 being fully inserted in the repair site; and

FIG. 8 is a schematic depicting the tissue repair system shown in FIGS. 2-5 being secured in the repair site.

### DEFINITIONS

As used herein, the singular forms “a,” “an,” and “the” include plural references unless the context clearly indicates otherwise. Thus, for example, reference to “an active agent” includes a single active agent as well as a mixture of two or more different active agents or a pharmaceutically acceptable salt, solvate, crystalline form, derivative, prodrug or analogue thereof; and reference to an “excipient” includes a single excipient as well as a mixture of two or more different excipients, and the like.

As used herein, the term “about” in connection with a measured quantity or time, refers to the normal variations in that measured quantity or time. as expected by one of ordinary skill in the art in making the measurement and exercising a level of care commensurate with the objective of measurement. In certain embodiments, the term “about” includes the recited number ±10%, such that “about 10” would include from 9 to 11, or “about 1 hour” would include from 54 minutes to 66 minutes.

The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that. In certain embodiments, the term “at least about” includes the recited number minus 10% and any quantity that is higher such that “at least about 10” would include 9 and anything greater than 9. This term can also be expressed as “about 10 or more.” Similarly, the term “less than about” typically includes the recited number plus 10% and any quantity that is lower such that “less than about 10” would include 11 and anything less than 11. This term can also be expressed as “about 10 or less.”

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illustrate certain materials and methods and does not pose a limitation on scope.

The terms “autologous” and “autograft” refer to tissue or cells which originate with or are derived from the recipient, whereas the terms “allogeneic” and “allograft” refer to cells and tissue which originate with or are derived from a donor of the same species as the recipient.

As used herein, the term “active agent” refers to any material that is intended to produce a therapeutic, prophylactic, or other intended effect, whether or not approved by a government agency for that purpose. This term with respect to a specific agent includes the pharmaceutically active agent, and all pharmaceutically acceptable salts, solvates, crystalline forms, derivatives, prodrugs or analogues thereof, where the salts, solvates, crystalline forms, derivatives, prodrugs or analogues are pharmaceutically active.

The term “pharmaceutically acceptable salt” as used herein refers to one or more salts of an active pharmaceutical ingredient (API). For example, pharmaceutically acceptable salts of basic APIs include hydrochloride, mesylate, hydrobromide, acetate and/or fumarate. Pharmaceutically acceptable salts for acidic APIs include sodium, calcium and/or potassium. A pharmaceutically acceptable salt may be chosen for a particular modified release fill formulation based on its aqueous solubility, stability, toxicity, absorption, manufacturability and/or other physicochemical and/or biological considerations.

The term “polymorphism” refers to the ability of an active ingredient (Al) to exist in more than one crystalline form and the term “polymorph” refers to at least one of the crystalline forms of an Al.

As used herein, the terms “therapeutically effective” and an “effective amount” refer to that amount of an active agent, or the rate at which it is administered, needed to produce a desired therapeutic result.

The term “subject” refers to a human or animal, that has demonstrated a clinical manifestation of a condition The term “subject” may include a person or animal (e.g., a canine) that is a patient being appropriately treated by a medical caregiver for a condition. In the present disclosure, a subject includes, but is not limited to, any mammal, such as human, non-human primate, mouse, rat, dog, cat, horse or cow.

The terms “treatment of” and “treating” include the administration of an active agent(s) with the intent to lessen the severity of a condition and/or a symptom.

The terms “prevention of” and “preventing” include the avoidance of the onset of a condition by a prophylactic administration of the active agent.

The terms “ambient” or “ambient conditions” as used herein refer to temperatures of about 15° C. to less than 37° C. at one (1) atmosphere of pressure.

The term “elevated temperature” as used herein refers to temperatures of about 37° C. and above at one (1) atmosphere of pressure.

The terms “flow,” “flowable,” “flowable fluid,” or “flowable liquid” as used herein refer to a fluid (e.g., a liquid) that has a viscosity of 100,000 cP or less at a temperature of 40° C. or 60° C. when the viscosity is determined using a Thermo Fischer Scientific HAAKE RheoStress 6000 parallel plate with shear stress at 10 Pa and an oscillation frequency of 1 Hz, at 40° C. or 60° C.

The term “particles” as used herein refers to granules, extrudates, powders, pellets, multi-particulates (e.g., coated sub-units, bi-layer sub-units, multi-layer sub-units), minitablets, microcapsules. cuboids, hexoids, rhomboids, spheres, microspheres, cylinders, ovals, fibers, and/or combinations thereof.

The term “w/w %” or “weight concentration” as used herein refers to the mass of the solute as a percentage of the total mass of the solution.

### DETAILED DESCRIPTION OF ILLUSTRATIVE EMBODIMENTS

Reference will now be made in detail to the various embodiments of the present disclosure illustrated in the accompanying drawings. Wherever possible, the same or like reference numbers will be used throughout the drawings to refer to the same or like features. It should be noted that the drawings are in simplified form and are not drawn to precise scale. In reference to the disclosure herein, for purposes of convenience and clarity only, directional terms such as top, bottom, left, right, above, below and diagonal, are used with respect to the accompanying drawings. Such directional terms used in conjunction with the following description of the drawings should not be construed to limit the scope of the present disclosure in any manner not explicitly set forth.

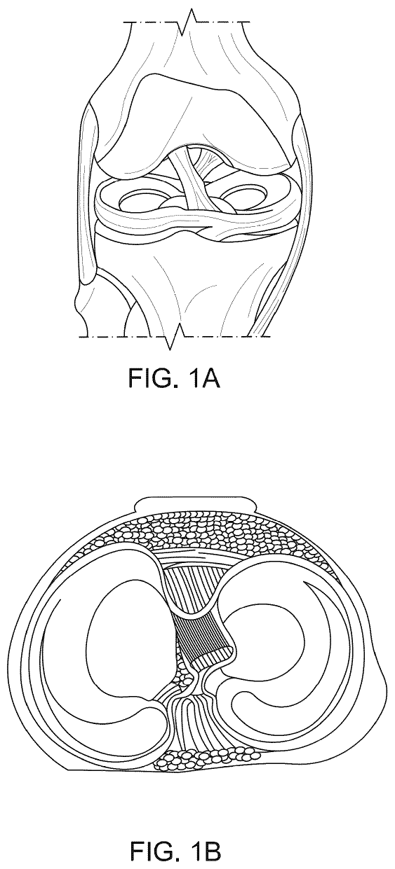

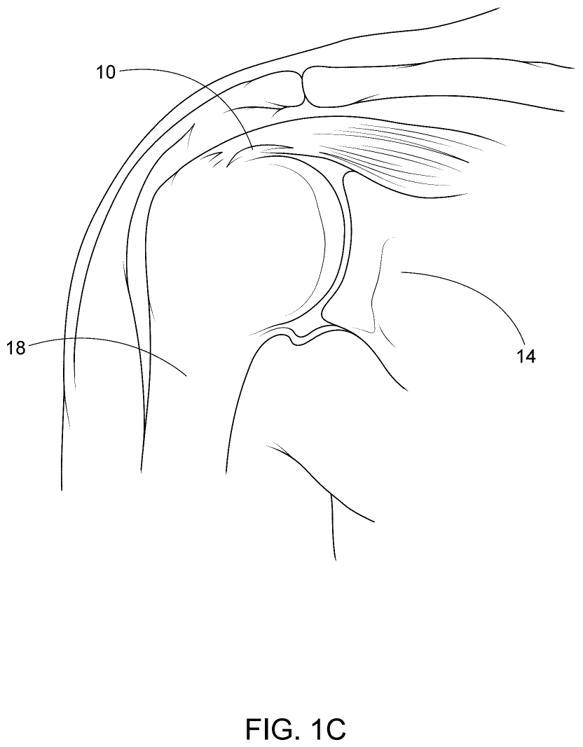

Referring to FIGS. 1-2, the repair system 100 includes a collagen implant 104 and one or more suture assemblies configured to secure the collagen implant in a repair site 10 at a tissue of interest. The collagen implant 104 is a compressible and biocompatible implant that is configured to absorb fluid, such as blood and/or blood components. The collagen implant 104 is configured to heal or repair tissue. In some embodiments, the collagen implant 104 may comprise a three-dimensional (3-D) scaffold for repairing the ruptured or torn tissue. Tissue here may include ligaments, cartilage, and tendons, such as an ACL, meniscus or rotator cuff, etc. Before or during implantation, blood or some other repair material, is injected or added to the collagen implant. This, in turn facilitates growth or regrowth of damaged or torn soft tissue. In one example, the collagen implant and repair material are used to repair or regrow rotator cuff injuries or tears. In another example, the collagen implant and repair material are used to repair or regrow meniscus tears. In some example,, the collagen implant may be secure in place at or adjacent a rupture or tear of the ACL while healing or growth of a damaged meniscus is observed.

The system 100 may further include a delivery device 116 configured to carry the collagen implant 104 for delivery at the repair site, a suture passer 120 configured to carry one or more assemblies 108, 112, and a syringe 124 configured to hold a fluid and inject the fluid into the collagen implant 104. In various embodiments, the collagen implant 104 is injectable from the delivery device 116.

The collagen implant 104 may be configured for securement to a first and second portion of a torn or ruptured tissue (e.g., ACL, meniscus, rotator cuff, etc.) in order to facilitate growth of the tissue at or near the tear or rupture. The one or more suture assemblies 108, 112 may be configured to pass through one or more of a first portion of the torn or ruptured tissue and a second portion of the tissue to secure the collagen implant in place on or along the tear or rupture of the tissue. The suture passer 120 may be configured to carry and pass: 1) the first suture assembly through the first portion of the tissue and the collagen implant, and 2) the second suture assembly through the second portion of the tissue and the collagen implant.

In embodiments, the system 100 may further include a syringe 124 configured to hold a fluid and inject the fluid into the collagen implant 104. In some embodiments, the fluid is blood. The delivery device 116 of system 104 may have the collagen implant 104 and the one or more suture assemblies 108, 112 preloaded into the delivery device 116. In an alternative embodiment, the collagen implant 104 and the one or more suture assemblies 108, 112 are provisioned outside of the delivery device 116, but are insertable through the delivery device.

The collagen implant 104 is formed from a self-assembly of collagen fibers, substantially free of cross-linking agents, and is absorbent. In some embodiments, the collagen implant 104 is injected into the delivery site in slurry or liquid form. The collagen implant 104 is then configured to self-assemble into a semi-solid or solid structure at or near the tear or rupture of the tissue. The term “self assembly” as used herein includes the self-aggregation of collagen fibers into higher-order structures such as ropelike structures, fibrils, microfibrils, fibers, fascicles, felt-like networks, for example, driven by molecular interactions. However, in embodiments, the self-assembled structures are substantially free (e.g., about 95% to about 100%) of cross-links.

The collagen implant 104 includes a structural integrity sufficient to receive therethrough one or more sutures for securement to the ruptured or damaged tissue. The collagen implant 104 is configured to interact with the subject's body to develop a network of capillaries, arteries, and veins at the tissue tear or rupture. Well-vascularized connective tissues heal as a result of migration of fibroblasts into the collagen implant 104. The methods and systems of the present disclosure establish a link between the damaged tissue, either by encircling the torn tissue or connecting with it, fostering the mending process of the ruptured or torn tissue while preserving its integrity and structure. The collagen implant may function either as an insoluble or biodegradable regulator of cell function or as a delivery vehicle of a supporting structure for cell migration or synthesis, and may provide a network or structure to facilitate cell in growth and vascularization.

The collagen implant 104 may be configured to provide a connection between the ruptured or torn portions of the tissue and fibers, or form around the torn tissue, after injury, and encourages the migration of appropriate healing cells to form scar and new tissue. The collagen implant 104 may therefore be a substitute for a clot. The collagen implant 104 is thus implanted adjacent to ruptured or torn portions of tissues, between the ruptured or torn portions of the tissue, or wrapped around the tissue. In other embodiments, the collagen implant 104, in combination with the suture assemblies, is capable of forming a connection between the implant and bone, between soft tissue and bone, and/or being formed around the torn tissue such that the integrity and structure of the tissue is maintained. The collagen implant 104 is therefore designed to stimulate cell proliferation and extracellular matrix production in the space near or adjacent ruptured tissue and the gap between the ruptured ends of the tissue or the tear in the tissue site, thus facilitating healing and regeneration.

The collagen implant 104 may be a compressible and expandable, biodegradable, porous material that has some resistance to degradation by fluids in the tissue repair site, (for example, synovial fluid). The collagen is soluble, e.g., acidic or basic. In the illustrated embodiment, the collagen implant 104 is a collagen implant comprising a self-assembly of interconnected collagen fibers that does not include any cross-linking agents.

Collagen implants 104 according to embodiments herein may have a size and shape chosen to complement the specific anatomical tissue present. The collagen implant 104 may take several forms, such as a generally cylindrical shape, a planer sheet or laminate, or a plurality of flowable particles. In some embodiments, the collagen implant is injected at the repair site and then self assembles into a solid or semi-solid structure that conforms to the shape of the tissue (e.g., to fill the tear or rupture, to simulate a clot, etc.). For example, the collagen implant may be particles in the form of a powder, flakes, and/or chunks, configured for insertion or injection at the repair site. In one or more embodiments, the particles may have a mean particle size D₅₀ of less than about 70 mm, or about 0.005 μm to about 70 mm, about 1.0 μm to about 50 mm, about 5.0 mm to about 25 mm, about 500 μm to about 2,000 μm, or any individual value or sub-range within these ranges.

In at least one embodiment, the collagen implant has a particle size suitable to be expelled through a standard male luer-lock opening, the opening having an inner diameter of approximately 2.0 mm (per ISO594-2 (1998) or ISO 80369-1 (2018)). In one or more embodiments, the implant may be formed of particles, also referred to herein as a dry flowable solid, having a particle size distribution D₅₀ of less than about 2 mm, or a mean size of about 0.005 μm to about 1.950 mm, or about 0.1 μm to less than about 2 mm, or about 1 μm to about 1 mm, or any individual value or sub-range within these ranges. In various embodiments, the collagen implant 104 (or each of the plurality of particles) may have a pore size of greater than about 10 μm, greater than about 10 μm to about 1,000 μm, or an individual value or sub-range within these ranges.

In various embodiments, the collagen implant may have a lower surface, an upper surface opposite the lower surface, and a side wall that extends from the lower surface to the upper surface. The side wall defining an outer perimeter that is shaped to fit between a femur and a tibia and along the tear of the tissue (e.g., ACL, meniscus) when positioned adjacent to the tissue.

In one or more embodiments, the collagen implant 104 includes at least one of: one or more collagen, one or more salt, one or more electrolyte, and/or one or more of a glycosaminoglycan. The collagen implant 104 may be Type I collagen, the implant may include other collagen types. For example, in another embodiment, the collagen may be type II, III, IV, V, IX or X. Type I collagen is predominantly found in bone, skin (e.g., in sheet-like structures), and tendon (e.g., in rope-like structures). Type I collagen is densely packed and provides structure to skin, bones, tendons and ligaments. Type I collagen is further typified by its reaction with the protein core of another connective tissue component known as a proteoglycan. Signaling regions that facilitate cell migration are present in Type I collagen. Type II collagen is found in elastic cartilage and provides joint support. Type III collagen may be found in the skin's middle layer (i.e., the dermis), muscles, and blood vessels. Type IV collagen is a thin layer of tissue that supports cells in the kidneys, lungs, intestines, and eyes. Type V collagen may be found in hair and cell surfaces. Type X collagen is found in bones and cartilage and is suitable to support the regulation of matrix mineralization, cartilage development, and tissue remodeling during growth and following injuries.

In embodiments, the collagen may be heterotrimeric, tropocollagen, atelocollagen, fibrillar collagen, or combinations thereof. In some embodiments, the collagen is present in various forms. For example, the collagen may be tropocollagen (e.g., homotrimeric collagen or heterotrimeric collagen), atelocollagen, fibrillar collagen, or combinations thereof. Homotrimeric collagen is comprised of collagen molecules having three identical polypeptide chains. Heterotrimeric collagen also is comprised of collagen molecules having three polypeptide chains, but at least two of the chains differ. Atelocollagen is comprised of collagen molecules that have been purified to remove telopeptide regions known to cause an immune response. Fibrillar collagen is comprised of three chain polypeptide helixes assembled into fibrils; fibrillar collagens aide in the development, growth and maintenance of tissues and organs.

In one or more embodiments, the collagen is Type I, heterotrimeric collagen having two α1 (I) chains, and one α2(I) chain. The α1(I) chains may be at least about 100 nm to about 500nm long, or about 100 nm to about 500 nm long, about 200 nm to about 400 nm long, about 300nm to about 350 nm long, or any individual value or sub-range within these ranges. In some embodiments, the collagen may comprise tropocollagen and/or atelocollagen and is free of fibrillar collagen in order to reduce the antigenicity of the material.

The collagen may be derived from a source tissue of any mammal species. For example, the collagen may be derived from rat, pig (porcine), cow (bovine), or human tissue. Tendons, ligaments, muscle, fascia, skin, cartilage, tail, or any source of soft collagenous tissue are useful. In various embodiments, the collagen is derived from a bovine tissue (e.g., bovine knee tendons and ligaments, bovine cadaveric knee tissue, bovine elbow tendons and ligaments, etc.). In some embodiments, the collagen is obtained from autologous cells. For example, the collagen may be derived from a patient's cultured fibroblasts. The collagen may then be used in that patient or other patients.

In various embodiments, the collagen is not cross-linked. Although cross-linking may improve mechanical strength of the implant materials, some cross-linking agents may cause cytotoxicity (e.g., glutaraldehyde) and/or foreign body reactions. Calcification and/or biodegradation also may occur with the use of glutaraldehyde. In some embodiments, bone regeneration is impacted with the use of some cross-linked collagen membranes as compared to non-cross-linked counterparts. Cell adhesion and proliferation may be altered by cross-linking, which may impact biocompatibility.

According to various embodiments, collagen implants 104 as described herein comprise collagen in an amount of about 40 w/w % to about 70 w/w %, a buffer (as described herein) in an amount of about 15 w/w % to about 30 w/w %, a first salt in an amount of about 2 w/w % to about 5 w/w %, a second salt in an amount of about 2 w/w % to about 5 w/w %, an electrolyte in an amount of about 5 w/w % to about 11 w/w %, and glycosaminoglycan in an amount of less than about 2 w/w %, or about 0.001 w/w % to less than about 2 w/w %, or any individual value or sub-range within these ranges. In some embodiments, the buffer comprises HEPES. In one or more embodiments, the first and second salt are independently chosen from calcium and sodium. In various embodiments, the electrolyte may be chloride.

In various embodiments, collagen implants 104 have a collagen content of greater than 400 mg/g, about 400 mg/g to about 10,000 mg/g, or any individual value or sub-range within these ranges. Collagen implants according to embodiments herein may include DNA in an amount of less than 50,000 ng/g, about 5 ng/g to less than about 50,000 ng/g, or any individual value or sub-rang within these ranges. Collagen implants may include one or more GAGs in an amount of greater than 100 μg/g, greater than about 100 μg/g to about 8,000 μg/g, or any individual value or sub-range within these ranges. Collagen implants may include a phospholipid having a phospholipid count of less than 3,000 μM/g, about 1 μM/g to less than about 3,000 μM/g, or any individual value or sub-range within these ranges. In some embodiments, collagen implants include pepsin in an amount of less than 12.5 mg/g, about 0.1 mg/g to less than about 12.5 mg/g, or any individual value or sub-range within these ranges. In some embodiments, the collagen implants comprises at least one of: a collagen content of greater than 400 mg/g, a GAG content of greater than 100 μg/g, a DNA content of less than 50,000 ng/g, a phospholipid count of less than 3,000 μM/g, or a pepsin content of less than 12.5 mg/g.

Collagen implants 104 according to embodiments herein may include one or more salts. Some salts are beneficial to the healing process and control infection when the collagen implant, formed from the collagen slurry, is implanted and biodegrades within the subject. Salts also may be used to produce desirable properties in the implant such as porosity, surface attraction, biomechanical support, biocompatibility, and/or antimicrobial properties. Salts that may be incorporated into the collagen slurry include, but are not limited to, calcium containing salts, magnesium containing salts, sodium containing salts, calcium chloride, magnesium chloride, sodium chloride, or combinations thereof. In some embodiments, the salt comprises sodium. The collagen slurry may contain one or more salt in an amount of about 2 w/w % to about 5 w/w %, or any individual value or sub-range within this range.

Collagen implants 104 as described herein may further include one or more electrolytes. Electrolytes play a role in surface modification of the collagen implant to promote bone integration and/or improve biocompatibility. The composition of the electrolyte in collagen implants made from the described collagen slurries can modify the properties of the implant surface to reduce inflammation and promote healing. Suitable electrolytes for collagen slurry compositions include, but are not limited to chloride, nitrate, sulfate, silicate, phosphate, aluminate, sodium tetraborate, or combinations thereof. In some embodiments, the electrolyte comprises chloride. The one or more electrolyte may be present in the collagen slurry in an amount of about 5 w/w % to about 11 w/w %, or any individual value or sub-range within this range.

In various embodiments, the collagen implant 104 contains one or more glycosaminoglycans (GAGs). GAGs are long chain polysaccharides that promote implant biocompatibility and tissue integration. Suitable GAGs for implants configured for injection according to embodiments herein include, but are not limited to heparan sulfate, chondroitin sulfate, dermatan sulfate, hyaluronic acid, keratan sulfate, or combinations thereof. GAGs may be present in the implants according to embodiments herein in an amount of less than about 5 w/w %, less than about 2 w/w %, less than about 1 w/w %, or about 0.001 w/w % to less than about 2 w/w %, or any individual value or sub-range within these ranges. In some embodiments, the collagen slurries have a GAG content of greater than about 100 μg/g, greater than about 100 μg/g to about 1,000 μg/g, or any individual value or sub-range within these ranges.

In various embodiments, the collagen implant 104 includes collagen in an amount of about 40 w/w % to about 70 w/w %, at least one salt in an amount of about 2 w/w % to about 5 w/w %, at least one electrolyte in an amount of about 5 w/w % to about 11 w/w %, or glycosaminoglycan in an amount of less than about 2 w/w %, or about 0.001 w/w % to less than about 2 w/w %, or any individual value or sub-range within these ranges. The term “w/w %” or “weight concentration” as used herein refers to the mass of the solute as a percentage of the total mass of the solution.

In some instances, the collagen implant 104 further includes a buffer. The buffer may be a zwitterionic buffer. Suitable buffers include, but are not limited to, includes 4-(2-hydroxyethyl)-1-piperazineethanesulfonic acid (HEPES), tris (hydroxymethyl) aminomethanc) (TRIS), (3-(N-morpholino) propanesulfonic acid) (MOPS), 3-[N-Tris(hydroxymethyl)methylamino]-2-hydroxypropanesulfonic acid (TAPSO), cacodylate SSC, succinic acid, glycine, sodium phosphate, sodium hydroxide, sodium chloride, magnesium chloride, acetic acid, thrombin buffer, L-ascorbic acid phosphate magnesium salt n-hydrate, MMP-1, MMP-2, MMP-3, MMP-9, RNAasc/DNAase, clastase, papain, citrate, sodium citrate, phosphate, saline, or combinations thereof.

According to various embodiments, the collagen implant 104 is combined with cells such as platelets, white blood cells, red blood cells, stem cells, fibroblasts, or combinations thereof. In some embodiments, the cells are derived from the subject to be treated. In other embodiments, the cells are derived from a donor that is allogeneic to the subject. Such materials may be added to the collagen composition prior to lyophilization and/or may be absorbed into the collagen implant (e.g., just prior to injection at the repair site).

In certain embodiments, platelets may be obtained as platelet rich plasma (PRP). PRP contains fibrin and platelets together with other plasma proteins found in the blood. PRP may further include white blood cells and red blood cells found in this preparation. In some embodiments, the PRP has a platelet concentration at least about 100 K/ml, at least about 300 K/ml, about 100 K/ml to about 10,000 K/ml, or any individual value or sub-range within these ranges. In embodiments, the platelet concentration is about 1.0 times to about 5.0 times the platelet concentration in the blood of the patient, or any individual value or sub-range within this range. In order to maintain the stability of the cells, a physiologic pH (i.e., about 6.2 to about 7.6) and a physiologic plasma osmolarity (i.e., about 280 osms/kg about 360 osms/kg) is used. In order to enhance the function of the PRP, PRP may be used within seven (7) days of being drawn from the patient or donor. The PRP may be isolated from the patient at time of surgery. It may be stored at about 20° C. to about 37° C. (room temp to body temp). However, isolation and storage of the cells may be achieved by any methods and for any length of time known in the art for maintaining the activity of the active components.

In a non-limiting example, platelets may be isolated from a subject's blood using techniques known to those of ordinary skill in the art. As an example, a blood sample may be drawn into a tube containing an anticoagulant, and the subsequent solution centrifuged at 700 rpm for 20 minutes and the platelet-rich plasma upper layer removed. Platelet density may be determined using a cell count as known to those of ordinary skill in the art. The platelet rich plasma may be mixed with collagen and applied to the patient.

In a non-limiting example, white blood cells may be isolated from a subject's blood using techniques known to those of ordinary skill in the art. As an example, a blood sample may be drawn into a tube containing an anticoagulant and centrifuged at 700 rpm for 20 minutes and the buffy coat containing white blood cells removed. The white blood cell density may be determined using a cell count as known to those of ordinary skill in the art. The white blood cells can be mixed with collagen and applied to the patient.

Mixing collagen and/or a collagen implant compositions as described herein with blood, platelet rich plasma, white blood cells, etc. may form a viscous paste or putty mixture, depending on the ratio. Suitable volumetric ratios include, but are not limited to about 1:1 to about 100:1 of the volume of the collagen or collagen implant composition to the volume of the blood, platelet rich plasma, white blood cells, etc., or any individual value or sub-range within this range. When collagen or a collagen implant composition in an amount of about 3 ml to about 1,000 ml, or any individual value or sub-range therein, is mixed with a volume of about 3 ml to about 10 ml of blood, platelet rich plasma, white blood cells, etc., the resulting mixture may be viscous enough to be expelled from the delivery device, but stable enough to stay within the confines of the anatomical fields into which it is injected. Suitable viscosities for the collagen, collagen composition, or for the mixture with blood, platelet rich plasma, white blood cells, etc., include, but are not limited to, about 1 cP to about 10 cP, about 1.2 cP to about 8 cP, about 3.5 cP to about 5.5 cP, or any individual value or sub-range within these ranges.

The collagen solution may also include any one or more of an anti-plasmin agent, an extracellular matrix (ECM) protein, other protein or enzyme inhibitors, antibodies to plasmin, antibodies to tissue plasminogen activator or urokinase plasminogen activator, non-toxic crosslinkers, calcium, dextrose or other sugars and cell nutrients in physiological concentrations. Anti-plasmin agents include but are not limited to antifibrinolytic enzymes such as plasminogen inactivator, plasminogen binding α₂ antiplasmin, non-plasminogen binding α₂ antiplasmin, α₂ macroglobulin, α₂ plasmin inhibitor, α₂ antiplasmin, and thrombin activatable fibrinolysis inhibitor. Other protein or enzyme inhibitors include but are not limited to anti-enzymatic proteins including inhibitors of collagenase, trypsin, matrix metalloproteinases, elastase and hyaluronidase. The ECM is composed of fibrillar and non-fibrillar components. The major fibrillar proteins arc collagen and clastin. The ECM includes for instance, diverse combinations of collagens, fibrinogen, proteoglycans, clastin, hyaluronic acid, and various glycoproteins including laminin, fibronectin, heparan sulfate proteoglycan, and entactin. Non-toxic crosslinkers include but are not limited to tissue transglutaminases, lysyl oxidase, fibrin, fibronectin, and reducible and non-reducible crosslink precursor molecules.

Collagen implants 104 according to embodiments herein may include one or more additional components, such as insoluble collagen, other extracellular matrix proteins (ECM), such as proteoglycans and glycosaminoglycans, fibronectin, laminin, entectin, decorin, lysyl oxidase, crosslinking precursors (reducible and non-reducible), clastin, elastin crosslink precursors, cell components such as, cell membrane proteins, mitochondrial proteins, nuclear proteins, cytosomal proteins, and cell surface receptors, growth factors, such as, PDGF, TGF, EGF, and VEGF, hydroxyproline, or combinations thereof. In one or more embodiments, the collagen implants 104 comprise one or more peptide, proteinase inhibitor, collagenase inhibitor, zinc ions, retinol, vitamin, steroid, hormone, cytokine, clotting factor, angiogenic protein, antiangiogenic protein, anti-protease protein, bone morphogenic protein, osteoinductive factor, fibronectin, cementum attachment extract, ketanserin, interlenkin-1, human alpha thrombin, an anti-inflammatory, or combinations thereof. According to various embodiments the anti-protease protein may include, but is not limited to alphalantitrypsin, the antiangiogenic protein comprises endostatin, angiostatin, or combinations thereof. In one or more embodiments, the growth factor comprises endothelial cell growth factor, epidermal growth factor, transforming growth factor-beta, insulin-like growth factor, platelet derived growth factor, periodontal ligament chemotactic factor, vascular endothelial growth factor, fibroblast growth factor, or combinations thereof. In embodiments, the hormone comprises human growth hormone, animal growth hormones, or combinations thereof. In at least one embodiment, the vitamin comprises vitamin A, vitamin B6, vitamin B9, vitamin B12, vitamin C, vitamin D, vitamin K, or combinations thereof.

In various embodiments, the anti-inflammatory includes a non-steroidal anti-inflammatory drug (NSAID), acetaminophen, an opioid, a cannabinoid, salicylic acid, lidocaine, or combinations thereof. Suitable NSAIDs include, but are not limited to ibuprofen, naproxen, diclofenac, celecoxib, mefenamic acid, etoricoxib, indomethacin, or combinations thereof. In some embodiments, the opioid comprises hydrocodone, oxycodone, morphine, oxymorphone, codeine, or combinations thereof. Suitable cannabinoids include, but are not limited to, tetrahydrocannabinol (THC), cannabidiol (CBD) and cannabinol (CBN). Other cannabinoids include for example, cannabichromene (CBC), cannabigerol (CBG) cannabinidiol (CBND), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM). As used herein THC, CBD, CBN, CBC, CBG, CBND, CBL, CBV, THCV, CBDV, CBCV, CBGV and CBGM refer to the decarboxylated form of the cannabinoid. Whereas THCA, CBDA, CBNA, CBCA, CBGA, CBNDA, CBLA, CBVA, THCVA, CBDVA, CBCVA, CBGVA and CBGAM (cannabigerolic acid monomethyl ether) refer to the corresponding acid form of the cannabinoid.

The collagen implants 104 described herein may have a porosity of at least about 50%, at least about 60%, at least about 70%, at least about 80%, about 50% to 99%, about 80% to 99%, or any individual value or sub-range within these ranges. In some embodiments, the collagen implant has a porosity of at least about 50%, at least about 60%, at least about 70%, at least about 80%, about 50% to 99%, about 80% to 99%, or any individual value or sub-range within these ranges. In some embodiments, the collagen implant has a porosity of less than about 10%, about 1% to about 10%, or any individual value or sub-range within these ranges.

### Meniscus and ACL Repair

Referring to FIGS. 1A-1B, the tissue repair system may be used to heal, regrow, or repair a torn or ruptured meniscus. A torn meniscus can manifest as a number of diverse types of tears and the extent and shape of the tear can vary. For example, a radial tear occurs within the avascular zone of the meniscus. A horizontal tear runs along the curved fibers of the meniscus. This type can occur in the vascular portion near the outer edge of the meniscus or be more centrally located. A flap tear occurs when part of the cartilage is folded or “peeled” back, causing it to catch on the joint. A bucket-handle tear occurs in the center of the meniscus. A partial meniscus tear has a partial thickness in depth and the meniscus still remains attached. On the other hand, a complete meniscus tear involves a full thickness tear, meaning that the tear has penetrated completely from the top to the bottom of the meniscus. The tissue repair system as described herein may be used for any of the above referenced tears.

More specifically, a tissue repair system for a tear or rupture of a meniscus may include a collagen implant as described according to embodiments herein having a lower surface 128, an upper surface 132 opposite the lower surface 128, and a side wall 136 that extends from the lower surface 128 to the upper surface 132. The side wall 136 defines an outer perimeter that is shaped to fit between a first and second bone along a tear on the tissue.

In this manner, the collagen implant 104 may be cylindrical in shape having a length L1 along a longitudinal axis 2, which extends from the lower surface 128 to the upper surface 132. The length L1 may range between 5 mm up 35 mm or more, depending on the tissue site. The collagen implant 104 may have a cross-sectional dimension D1, which extends along a latitudinal axis 4 and is perpendicular to the length L1. In some examples, the length L1 may be less than a cross-sectional dimension D1 of the implant 104. In such an example, the cross-sectional dimension D1 may be about 10 mm to about 30 mm and the length L1 may be less than about 10 mm. For example, the length L1 could be about 2 mm to about 10 mm. In cases where the length L1 is less than D1, the collagen implant 104 may suitable for meniscus repair because its shape could more or less correspond to the meniscal tissue dimensions, or be smaller but the same general shape. In another example, however, the collagen implant is a more elongated structure where the length L1 is greater than a cross-sectional dimension D1 of the of collagen implant 104. For example, cross-sectional dimension is about 20 mm to about 30 mm and the length L1 could be up to about 40 mm. In another example, the cross-sectional dimension D1 is about 25 mm. A cylindrically shaped scaffold is suitable for use with meniscus procedures (and rotator cuff procedures as described below). However, the size and shape of the collagen implant 104 can vary as needed for other anatomies and tissue sites.

The side wall may define an outer perimeter that is shaped to fit between a femur and a tibia and along the tear of the meniscus when positioned adjacent to the meniscus. In this example, the collagen implant may generally have a shape that matches the profile a meniscus, such as a disc shape. In such an example, a first portion of the implant may have a thickness that is greater than a thickness of a second portion of the implant that spaced laterally with respect to the first portion. As described above, the collagen implant is substantially free of cross-linking agents and includes the components described above.

In this instance, the collagen implant is configured for securement to a first and second portion of a meniscus in order to facilitate growth of meniscal tissue at or near the tear and may be positioned with the delivery device as described above. Such a tissue repair system includes one or more suture assemblies 108, 112 configured to pass through a) a first portion of the meniscus, b) a second portion of the meniscus, or c) both the first and second portions of the meniscus to secure the collagen implant in place on or along the tear of the meniscus. In one example, the collagen implant is injectable from the delivery device.

A method of repairing a meniscus may include forming a portal in a knee joint to access a meniscus having a rupture or tear. Next, a user may insert or inject a delivery device into the portal so that a distal end of the delivery device is positioned close to the meniscus. The user can then cause the collagen implant to exit the distal end of the delivery device in order to position it on or near the rupture or tear of the meniscus. The user can the secure, with one or more suture assemblies, the collagen implant in place at or near the rupture or tear of the meniscus. For example, the securing step may include threading a first suture assembly onto a first portion of the meniscus, and threading a second suture assembly onto a second portion of the meniscus.

In addition, in other example, the user may thread the collagen implant along or on at least one of the first suture assembly and the second suture assembly after the sutures are secured to the tissue. In other examples, the user may thread the collagen implant along or on at least one of the first suture assembly and the second suture assembly before the sutures are secured to the tissue. The suture assemblies and collagen implant can be preloaded in the delivery device and then ejected for placement at or on the meniscus. In some embodiments, securing the collagen implant in place at the rupture or tear of the meniscus includes threading a first suture assembly onto a first portion of the meniscus and threading a second suture assembly onto a second portion of the meniscus.

At some time during the procedure, the patient's blood may be injected into the collagen implant 104. For example, in an embodiment, the user (e.g., the surgeon, nurse, technician, etc.) injects a volume of blood into the collagen implant before the implant is placed at the tissue site. In another embodiment, the user injects a volume of blood into the collagen implant before the collagen implant exits the distal end of the delivery device.

Further described herein according to embodiments are methods for delivering a collagen implant as described herein to a ruptured or torn ACL. According to various embodiments of the disclosure, the methods for delivering a collagen implant to a meniscus may include delivering one or more implant at or near an ACL whether healthy, torn, or ruptured. The methods may include observing growth of the ACL and meniscal tissue following delivery and securement of the implant at or near a rupture or tear of the ACL and the meniscus.

The methods may include positioning a bone fixation device on a femur and coupling the collagen implant to the femur with the bone fixation device. One or more suture assemblies may be attached to the bone fixation device as described herein according to embodiments. The one or more suture assemblies may be secured to the ACL having the rupture or tear, for example, a first suture may be secured to a first portion of the torn or ruptured ACL and a second suture may be secured to a second portion of the torn or ruptured ACL. As described herein, a volume of blood may be injected into the collagen implant during the process of implantation (e.g., prior to insertion into the delivery device, within the delivery device, following ejection from the delivery device, etc.). The collagen implant may be threaded along or on the one or more suture assemblies so that the blood is positioned at or adjacent the rupture or tear of the ACL and a rupture or tear of a meniscus. In this embodiment, growth of the ACL and meniscal tissue may be observed at or near the rupture or tear of the ACL and a meniscus.

### Rotator Cuff

Referring to Figures 1C-1D, the tissue repair system is configured to repair a rotator cuff injury. A torn rotator cuff can manifest as a number of diverse types of tears. The extent and shape of the tear can vary. A partial rotator cuff tear has a partial thickness in depth and the rotator cuff still remains attached. On the other hand, a complete rotator cuff tear involves a full thickness tear, meaning that the tear has penetrated completely from the top to the bottom of the rotator cuff.

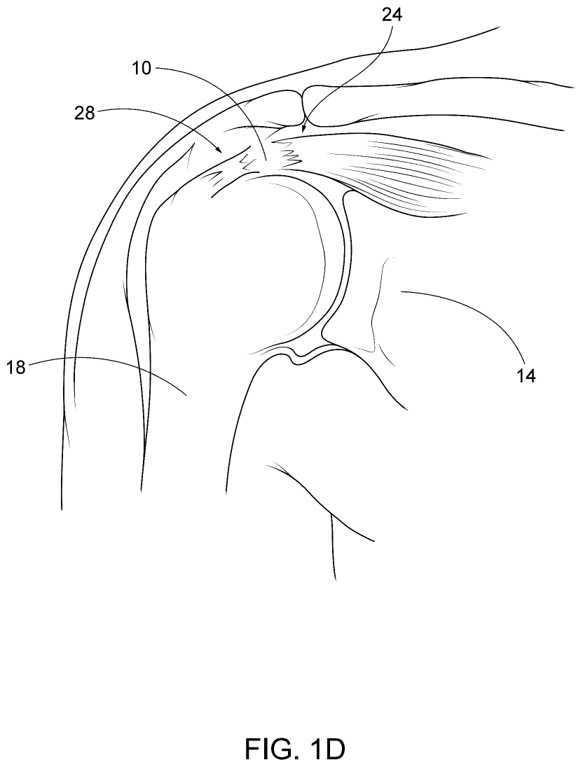

Referring to FIGS. 2-4, the collagen implant 104 may have a size and shape selected to complement the specific anatomical tissue present, such as a torn meniscus, ACL, or rotator cuff. In one example, the collagen implant 104 may be a generally elongated structure having a lower surface 128, an upper surface 132 opposite the lower surface 128, and a side wall 136 that extends from the lower surface 128 to the upper surface 132. The side wall 136 defines an outer perimeter that is shaped to fit between a first and second bone along a tear on the tissue.

In this manner, the collagen implant 104 is cylindrical in shape having a length L1 along a longitudinal axis 2, which extends from the lower surface 128 to the upper surface 132. The length L1 may the range of about 15 mm to about 35 mm. In one example, the length L1 is at least about 25 mm. The collagen implant 104 may have a cross-sectional dimension D1, which extends along a latitudinal axis 4 and is perpendicular to the length L1, that ranges from about 10 mm to about 30 mm. In one example, the cross-sectional dimension is from about 20 mm to 25 mm. In another example, the cross-sectional dimension D1 is about 22 mm. Thus, in a generally elongated scaffold, the length L1 is greater than the cross-sectional dimension D1. A cylindrically shaped scaffold is suitable for use with meniscus and rotator cuff procedures, as described below. However, the size and shape of the collagen implant 104 can vary as needed for other anatomies and tissue sites.

In this manner, the collagen implant 104 is cylindrical in shape having a length L1 along a longitudinal axis 2, which extends from the lower surface 128 to the upper surface 132. The length L1 may range between 15 mm and 35 mm. In one example, the length L1 is at least 25 mm. The collagen implant 104 may have a cross-sectional dimension D1, which extends along a latitudinal axis 4 and is perpendicular to the length L1, that ranges between 10 mm and 30 mm. In one example, the cross-sectional dimension is between 20 mm and 25 mm. In another example, the cross-sectional dimension D1 is about 22 mm. Thus, in a generally elongated scaffold, the length L1 is greater than the cross-sectional dimension D1. A cylindrically shaped scaffold is suitable for use with meniscus and rotator cuff procedures, such as described below. However, the size and shape of the collagen implant 104 can vary as needed for other anatomies and tissue sites.

The collagen implant 104 is hydrophilic and capable of absorbing plasma, blood, other body fluids, liquid, hydrogel, or other material the scaffold either comes into contact with or is added to the scaffold. The collagen implant 104 may be referred to hereinafter as a collagen scaffold, sponge, or collagen sponge.

In the illustrated embodiment, the collagen implant 104 is treated with a repair material prior to insertion into a repair site. As illustrated, the repair material is blood. Specifically, the collagen implant 104 is treated with 5-10 mL of autologous blood. The collagen implant 104 may be either soaked in the repair material or the repair material is injected into the collagen implant 104 prior to or during implantation into the repair site. In other examples, the repair material may be an autologous or allogeneic blood composition, plasma or other fluids either present within the repair site, added to the collagen implant 104 either before or after the collagen implant 104 is inserted into the repair site, or added into the repair site.

While a cylindrical shape is shown as suitable for certain meniscus and rotator cuff repairs, the collagen implant 104 may be any shape that is useful for implantation into and repair of tissue. The collagen implant 104, for instance, may be tubular, semi-tubular, a flat planar sheet, or a flat sheet rolled into a tube so as to define a hollow cavity. Other shapes suitable for the implant as known to those of ordinary skill in the art are also contemplated in the invention.

In addition, in alternative embodiments, the implant may include additional components to aid in healing, cellular ingrowth and vascularization. Such additional components may include proteins, including, but not limited to, hormones, cytokines, growth factors, clotting factors, anti-protease proteins (e.g., alpha1-antitrypsin), angiogenic proteins (e.g., vascular endothelial growth factor, fibroblast growth factors), antiangiogenic proteins (e.g., endostatin, angiostatin), and other proteins that are present in the blood, bone morphogenic proteins (BMPs), osteoinductive factor (IFO), fibronectin (FN), endothelial cell growth factor (ECGF), cementum attachment extracts (CAE), ketanserin, human growth hormone (HGH), animal growth hormones, epidermal growth factor (EGF), interleukin-1 (IL-1), human alpha thrombin, transforming growth factor (TGF-beta), insulin-like growth factor (IGF-1), platelet derived growth factors (PDGF), and fibroblast growth factors (FGF, bFGF, etc.), for therapeutic purposes. A lyophilized material is one that is capable of swelling when liquid, gel or other fluid is added or comes into contact with it.

The collagen implant 104 may be compressed prior to or during implantation into a repair site. A compressed implant allows for the implant to expand within the repair site. The collagen implant 104 may be lyophilized and/or compressed when placed in the repair site and expanded once in place. The expansion of the collagen implant 104 may occur after contact with blood or other fluid in the repair site or added to the repair site.

In another embodiment, the collagen implant 104 may be saturated or coated with a gel or hydrogel repair material prior to implantation into a repair site. Coating or saturation of the collagen implant 104 may case implantation into a relatively undefined defect arca as well as help to fill a particularly large defect area. In a preferred embodiment, the collagen implant 104 is treated with hydrogel. Examples of scaffolds and repair materials that may be used according to the present disclosure are found in U.S. Pat. No. 6,964,685 and U.S. Patent Application Nos. 2004/0059416 and 2005/0261736, the entire contents of each are herein incorporated by reference.

The biologic properties of cell infiltration rate and scaffold degradation may also be altered by varying the pore size, degree of cross-linking, and the use of additional proteins, such as glycosaminoglycans, growth factors, and cytokines in the collagen implant 104. In addition, collagen-based biomaterials can be manufactured from a patient's own skin, thus minimizing the antigenicity of the implant. However, preferable collage scaffolds do not include any cross-linking agents.

Continuing with FIGS. 1-4, in the illustrated embodiment, the collagen implant 104 is attached to the ruptured tissue via a first suture assembly 108 and a second suture assembly 112. The first suture assembly 108 is configured to pass through the collagen implant 104 and a first portion 144A of the ruptured tissue. The second suture assembly 112 is configured to pass through the collagen implant 104 and a second portion 144B of the ruptured tissue, wherein the first suture assembly 108 and the second suture assembly 112 secure the collagen implant 104 in place between a first bone and a second bone along the tear of the tissue.

In the illustrated embodiment, the suture assemblies 108, 112 may include a first suture and a second suture. The sutures are bioabsorbable, such that the subject is capable of breaking down the suture assemblies 108, 112 and absorbing them. The suture assemblies 108, 112 are also synthetic such that they may not be from a natural source. In other embodiments, the suture assemblies 108, 112 may be permanent such that the subject is not capable of breaking down the suture and they remain in the subject. The suture assemblies 108, 112 may be rigid or stiff, or may be stretchy or flexible. Examples of sutures include, but are not limited to, VICRYL™ polyglactin 910, PANACRYL™ absorbable suture, ETHIBOND® EXCEL polyester suture, PDS® polydioxanone suture and PROLENE® polypropylene suture. Sutures are available commercially from manufacturers such as MITEK PRODUCTS division of ETHICON, INC. of Westwood, Mass.

The system 100 may further include a delivery device 116 configured to carry the collagen implant 104 for delivery at the rupture or tear of the tissue. The delivery device 116 may have an elongated body, a proximal end, a distal end opposite the proximal end, and a channel that extends from the distal end toward the proximal end, the channel defining a cross-sectional dimension that is shaped and sized to carry the collagen implant. The delivery device may include a moveable rod in the channel and is configured to eject the collagen implant 104 from the distal end of the delivery device into tissue.

The collagen implant 104 may be compressed prior to or during implantation into the repair site. A compressed implant allows for the implant to expand within the repair site. The collagen implant 104 may be lyophilized and/or compressed when placed in the repair site and expanded once in place. The expansion of the collagen implant 104 may occur after contact with blood or other fluid in the repair site or added to the repair site. The delivery device 116 may be configured to carry the collagen implant 104 in the compressed state for delivery at or near the tear of the rotator cuff.

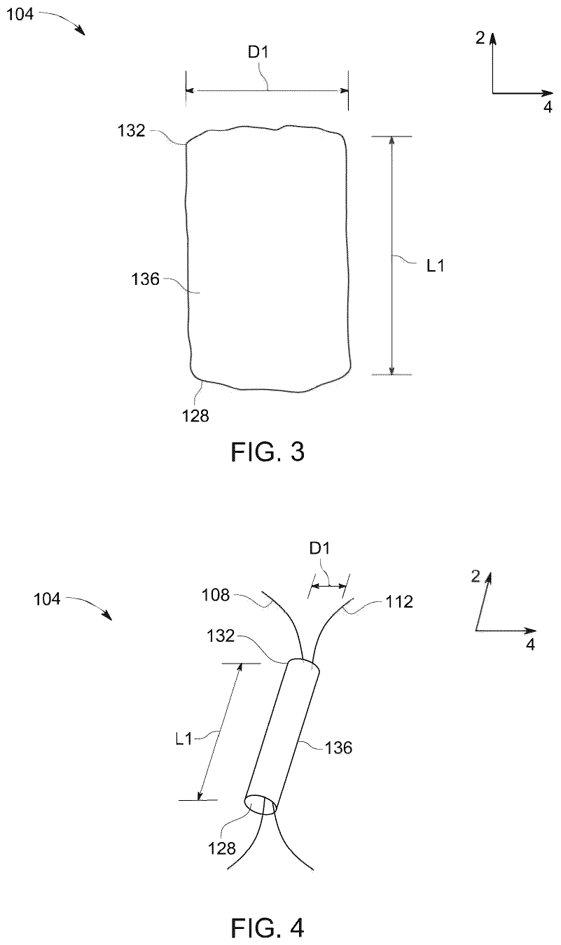

Referring to FIG. 5, a delivery device 116 is configured to carry the collagen implant 104 in a compressed state for delivery at the site of the tear of the tissue. The delivery device 116 has an elongated body 156, a proximal end 160, a distal end 164 opposite the proximal end 160, and a channel 168 that extends from the distal end 164 toward the proximal end 150. The channel 168 defines a cross-sectional dimension that is shaped and sized to carry the collagen implant 104 in the compressed state. The delivery device 116 includes a moveable rod 172 in the channel 168 and configured to eject the collagen implant 104 from the distal end 164 of the delivery device 116 into the tissue site.

In the illustrated embodiment, the delivery device 116 is a syringe. The syringe may hold the suture assemblies 108, 112 and the collagen implant 104 in place within the elongated body 156 of the syringe. The syringe may include a plunger configured to push the suture assemblies 108, 112, and the collagen implant 104 into a repair site such that the collagen implant 104 is positioned along the suture assemblies 108, 112 and adjacent to at least one ruptured end of the tissue. In alternative embodiments, the delivery device 116 may include a cannula, such as an arthroscopic cannula, a container, and a pressure pump. In another embodiment, the delivery device 116 may further include a guiding suture that extends out of the distal end of the elongated body 156, where the guiding suture is configured to pull and position the suture assemblies 108, 112 and the collagen implant 104 into the repair site. In yet another embodiment, the system may be inserted into the repair site without the use of arthroscopic equipment and instead through an open surgical procedure.

According to further embodiments of the disclosure are methods for delivering a collagen implant 104 to or near a torn or ruptured rotator cuff. The methods may include positioning a bone fixation device on a humerus. In some embodiments, the methods include attaching a first fixation device to the humerus. A second fixation device may be attached to the rotator cuff. In embodiments, the collagen implant 104 may be coupled to the humerus with the bone fixation device. One or more suture assemblies may be attached to the bone fixation device as described herein according to embodiments. In embodiments, the methods include connecting one or more suture assemblies to the first and second fixation devices. The collagen implant 104, optionally injected with blood may be placed along or the one or more suture assemblies. The collagen implant 104 may then be positioned between the ruptured or torn ends of the rotator cuff.

Referring to FIGS. 5-8, aspects of the invention relate to methods of repairing a ruptured or torn lateral or medial meniscus. A portal in a knee joint is formed to access a lateral or medial meniscus. The delivery device 116 is inserted into the portal so that a distal end of the delivery device 116 is positioned close to the lateral or medial meniscus. The collagen implant 104 is manipulated such that the collagen implant 104 exits the distal end of the delivery device 116 in order to position it proximate the rupture or tear of the lateral or medial meniscus. The collagen implant 104 is then secured in place at the rupture or tear of the lateral or medial meniscus via the suture assemblies 108, 112. A volume of blood is then injected into the collagen implant 104.

Continuing with FIGS. 5-9, aspects of the invention further relate to methods of repairing a meniscus by repairing a ruptured or torn ACL, at a repair site 240. In some embodiments, an implant 210, and sutures 224A, 224B are inserted into the repair site 240 of the torn meniscus via a delivery device 230.

An example of a ruptured anterior cruciate ligament is depicted in FIG. 5. The anterior cruciate ligament (ACL) 202 is one of four strong ligaments that connects the bones of the knee joint. The function of the ACL is to provide stability to the knee and minimize stress across the knee joint. It restrains excessive forward movement of the lower leg bone, the tibia 206, in relation to the thigh bone, the femur 204, and limits the rotational movements of the knee.

The anterior cruciate ligament 202 is ruptured such that it no longer forms a connection between the femur bone 204 and the tibia bone 206. The resulting ends of the ruptured ACL 202 may be of any length. The ends may be of a similar length, or one end may be longer in length than the other. The end on the femur 204 includes the femoral ACL stump 207. The end on the tibia 206 includes a tibial stump 209. In some instances, it is believed that a repair is desirable when the tibial stump length SL is less than about 75% of the effective ligament length LL but greater than 5% of a total length LL of the ACL. The total length of the ACL is considered to be the length of ligament from femoral footprint to the tibial footprint along a linear axis.

The knee joint includes tibial spines on the tibia 206 and the intercondylar notch of the femur 204. In some instances, the methods as described herein may include performing a notchplasty of the intercondylar notch of the femur to provide space for larger ligament to form after surgical repair using a scaffold. Such a notchplasty improves the size of the healing ligament, specifically resulting in a larger cross-sectional area of the ligament. As the mechanical strength of a ligament, and subsequently its ability to maintain the distance between the femur and tibia, is directly correlated with its cross sectional area, enlarging the notch with a notchplasty can help make a stronger repaired ACL and has been found by the inventors to be beneficial in ACL repair using a scaffold as described in the present disclosure.

Aspects of the invention provide methods of repairing the ruptured ligament 202 involving drilling one or more holes 244 at or near the repair site 240 of the ruptured ligament 202. A bone at or near a repair site is one that is within close proximity to the repair site and can be utilized using the methods and devices of the invention. For example, the bone at or near a repair site of a torn anterior cruciate ligament is the femur 204 bone and/or the tibia 206 bone. The hole 244 can be drilled into a bone using a device such as a Kirschner wire (for example a small Kirschner wire) and drill, or microfracture pics or awls. One or more holes may be drilled into a bone surrounding the repair site 240 to promote bleeding into the repair site 240. The repair can be supplemented by drilling holes into the surrounding bone to cause bleeding. Encouraging bleeding into the repair site may promote the formation of blood clots and enhance the healing process of the injury.

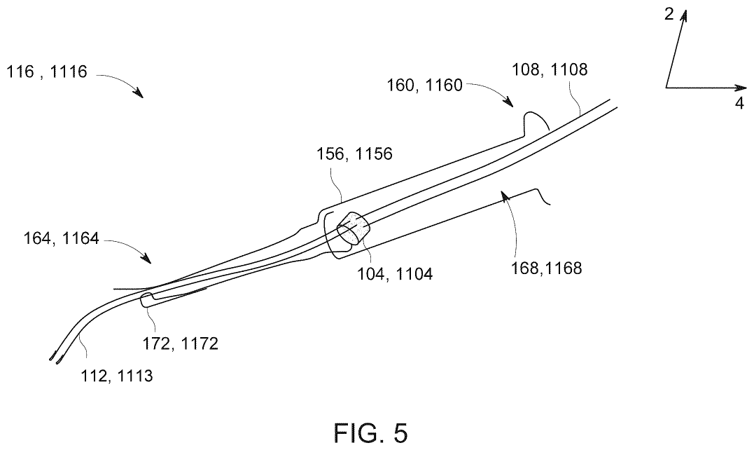

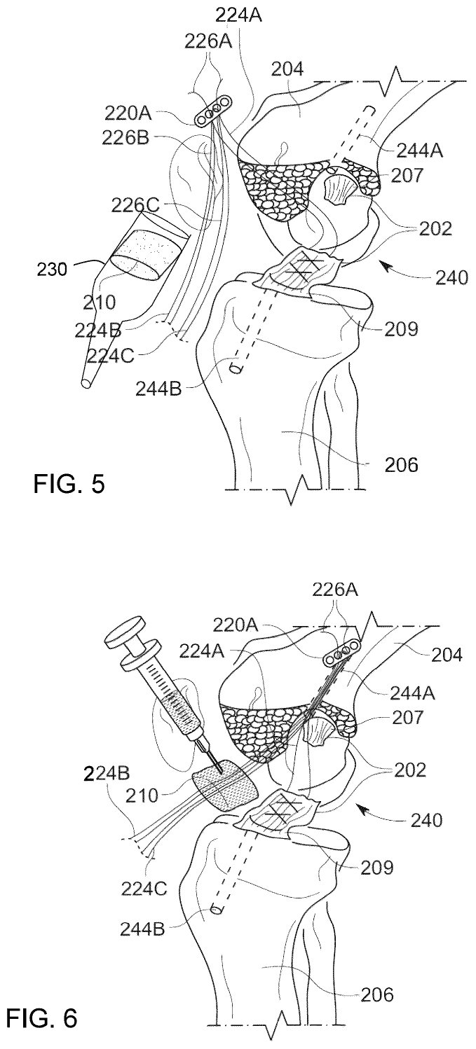

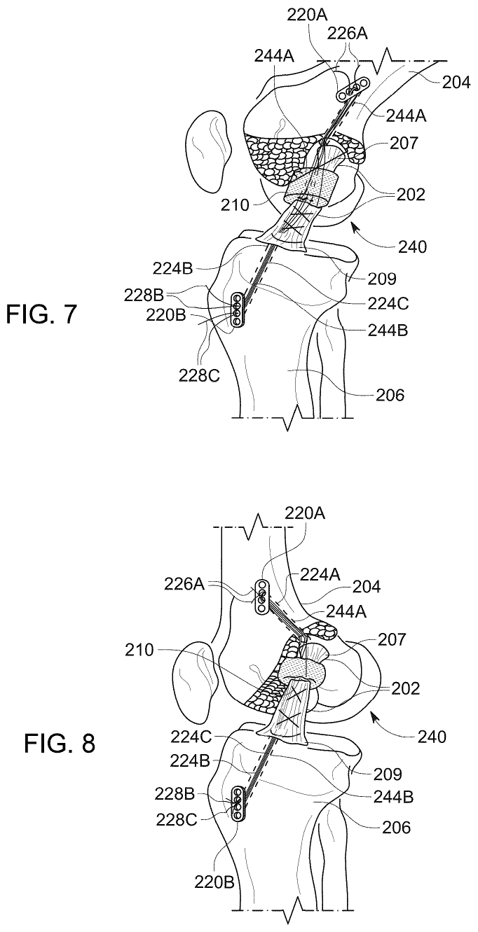

In FIG. 5, holes 244A, 244B are drilled into the femur 204 and the tibia 206, respectively, at the repair site 240. The hole 244A may be additionally referred to hereinafter as the femoral tunnel 244A and the hole 244B may be additionally referred to hereinafter as the tibial tunnel 244B A first suture 224A is placed through the tibial stump 209 using a whip-stitch. The first suture 224A is attached via the first end 226A to a first fixation device 220A. A second suture 224B and a third suture 224C are coupled to the fixation device 220A at respective first ends 226B, 226C. The fixation device 220A is subsequently passed through the femoral tunnel 244A and coupled to the femur 204. In FIG. 6, the implant 210 is loaded onto the second and third sutures 224B, 224C. The implant 210 is then injected with repair material as described. The implant 210 and the second and third sutures 224 may be inserted into the repair site 240 via the delivery device 230. In FIG. 7, the free ends 228B, 228C of the second and third sutures 224B, 224C are passed through the tibial tunnel 244B and are coupled to a second fixation device 220B coupled to the tibia 206. The implant 210 is then positioned between the two ends of the torn ACL 2. In FIG. 8, the knee is extended and the sutures 224A, 224B, 224C, and the fixation devices 220A, 220B are secured. As described here and in the embodiments below, the first and second fixation devices may be anchors. In another embodiment, the first and second fixation devices may be screws, barbs, or extracortical buttons.

In another embodiment, the implant 210 may be indirectly coupled to the first and second fixation devices 220A, 220B and held in position in the repair site 240 by additional sutures 224. In addition, in another embodiment, any of the first, second, or additional sutures 224A, 224B, . . . 224n may be attached to one or both ends of a ruptured ligament 202 by their first ends 226A, 226B, . . . 226n and/or their second ends 228A, 228B, . . . 228n. Furthermore, in another embodiment, additional fixation devices 220 and sutures 224 may be directly or indirectly attached to either the tibia bone 6 or the femur bone 204 to secure the implant 210 in position. In alternative embodiments, the implant 210 may be attached to the femur bone 204 directly or indirectly.

In yet another embodiment, a tibial tunnel is drilled in a tibia. A femoral tunnel is drilled in a femur so that the femoral tunnel and tibia tunnels are aligned. An extracortical button is placed on a femoral cortex. A first and second set of sutures are attached to the button and pass through the femoral tunnel. The first set of sutures are secured onto the ACL having a rupture or tear. A collagen implant is threaded along or on the second set of sutures. A volume of blood is injected into the collagen implant so that the blood is positioned at or adjacent the rupture or tear of the ACL and a rupture or tear of the meniscus. The first and second set of sutures, attached to a second button, are passed through the tibial tunnel. The second button is secured in place on the tibial cortex. Growth of ACL tissue and meniscus at or near a rupture or tear of the ACL and the meniscus is observed. This includes obtaining and analyzing an MRI image of the ACL tissue and meniscus at the tissue site and analyzing the MRI image to identify growth of the ACL tissue and meniscus.

In yet another embodiment, a first set of sutures and a second set of sutures are coupled to a bone fixation device. A first portion of the first set of sutures is secured to a ruptured or torn anterior cruciate ligament. The bone fixation device is positioned on a surface of the femur so that the first and second sets of sutures extend through a femoral tunnel. A collagen implant is positioned along the second set of sutures. A volume of blood is injected into the collagen implant so that the blood is proximate the ACL and a lateral or medial meniscus. The second set of sutures is secured to a surface of the tibia so that the second set of sutures extending through a tibial tunnel with a second bone fixation device. Growth of an ACL tissue and meniscus at or near a rupture or tear of the ACL and the meniscus is observed. This includes obtaining and analyzing an MRI image of the ACL tissue and meniscus at the tissue site and analyzing the MRI image to identify growth of the ACL tissue and meniscus.

In yet another embodiment, a first portion of the first set of sutures are secured to a ruptured or torn anterior cruciate ligament. A second portion of the first set of sutures are secured relative to the femur such that the first set of sutures extends from the ruptured or torn anterior cruciate ligament and through into the femoral tunnel to the bone fixation device. A first portion of the second set of sutures are secured relative to the tibia so that a suture tail of the second set of sutures extends through a tibial tunnel into the region near the anterior cruciate ligament. A second portion of the second set of sutures are secured relative to a femur at the bone fixation device so that the second set of sutures extends into a femoral tibia tunnel of the tibia alongside or adjacent the anterior cruciate ligament. A volume of blood is injected into a collagen implant. The collagen implant, with the volume of blood absorbed therein, is threaded along the second set of suture to a location adjacent the rupture or torn portion of the anterior cruciate ligament. Blood from the collagen implant is permitted to emanate from the collagen implant into the region near the anterior cruciate ligament and a tear or rupture in either or both of a lateral meniscus and a medial meniscus. Healing of the rupture or tear of ACL and the lateral meniscus or medial meniscus is observed. Growth of ACL tissue and meniscus tissue at or near a rupture or tear of the ACL and the meniscus is further observed. This includes obtaining and analyzing an MRI image of the ACL tissue and meniscus at the tissue site and analyzing the MRI image to identify growth of the ACL tissue and meniscus.

Referring to FIG. 8, the injury is a torn rotator cuff. A method for rotator cuff repair utilizing the implant 210 is described below. The method includes attaching a first fixation device 1220A to a humerus 1204 at a location other than an insertion site of the rotator cuff tendon, attaching a second fixation device 1220B to the tendon at a location remote from the injury site, and connecting one or more flexible constructs 1224 to the two fixation devices. The method further includes placing the implant 210 along the one or more flexible constructs 1224. The method further includes delivering the implant 210 and the one or more flexible constructs 1224 to the rotator cuff such that the implant 210 rests between torn ends of the rotator cuff tendon.

In some embodiments, the flexible construct 1224 is a suture. In some embodiments, the suture is absorbable. In other embodiments, the suture is nonabsorbable. In some embodiments, the suture configuration itself is used as the fixation method in the tendon. In one embodiment, the collagen implant 210 may be threaded along or on a second, separate flexible construct. The collagen implant 210 is positioned between a ruptured portion of the rotator cuff via the flexible constructs.

The method further includes attaching the first fixation device 1220A and the second fixation device 1220B only indirectly to the collagen implant 210. A volume of blood is injected into the implant 210.

Referring back to FIG. 5, a delivery device 1116 is configured to carry the collagen implant 104 in a compressed state for delivery at the site of the tear of the rotator cuff. The delivery device 1116 has an elongated body 1156, a proximal end 1160, a distal end 1164 opposite the proximal end 1160, and a channel 1168 that extends from the distal end 1164 toward the proximal end 1150. The channel 1168 defines a cross-sectional dimension that is shaped and sized to carry the collagen implant 104 in the compressed state. The delivery device 1116 includes a moveable rod 1172 in the channel 1168 and configured to eject the collagen implant 104 from the distal end 1164 of the delivery device 1116 into the tissue site.

In the illustrated embodiment, the delivery device 1116 is a syringe. The syringe may hold the suture assemblies 108, 112 and the collagen implant 104 in place within the elongated body 156 of the syringe. The syringe may include a plunger configured to push the suture assemblies 108, 112, and the collagen implant 104 into a repair site such that the collagen implant 104 is positioned along the suture assemblies 108, 112 and adjacent to at least one ruptured end of the meniscus. In alternative embodiments, the delivery device 116 may include a cannula, such as an arthroscopic cannula, a container, and a pressure pump. In another embodiment, the delivery device 116 may further include a guiding suture that extends out of the distal end of the elongated body 156, where the guiding suture is configured to pull and position the suture assemblies 108, 112 and the collagen implant 104 into the repair site. In yet another embodiment, the system may be inserted into the repair site without the use of arthroscopic equipment and instead through an open surgical procedure.

Continuing with FIG. 8, in the illustrated embodiment, the implant 210 is placed on the flexible construct 1224 so that the implant 210 rests between the torn ends of the rotator cuff tendon without mechanically attaching the implant 210 to the rotator cuff tendon. In some embodiments more than one flexible construct 1224 is placed between the first and second fixation devices 1220A, 1220B. In other embodiments more than one implant 210 is loaded onto the flexible constructs 1224 so that the implants 210 rest between the torn ends of the rotator cuff tendon without mechanically attaching the implant 210 to the rotator cuff tendon or to each other.

### EXAMPLES

### Example 1

### Preparation of a Collagen Implant

Surgical rotator cuff repair is conducted when non-surgical efforts do not yield the desired results. In principle during the surgery, an implant is placed in, on or over the injury site to bridge the tear in the rotator cuff. The approach is either open, mini-open or arthroscopic, each with its own variation in surgical technique. Known challenges with rotator cuff repair relate to implant deployment and placement, due to confined space, especially in mini-op and arthroscopic repair.

The collagen implants investigated here included injectable powders, injectable pieces, and semi-solid or solid implants of various shapes. Collagen implants were prepared utilizing the manufacturing process described in U.S. Pat. No. 11,826,489, which is incorporated by reference herein in its entirety.

Extraction process: To be usable for surgery in human or veterinary medicine, all elements that the receiving host could recognize as foreign were removed. This was achieved via a series of steps, including:
1. Salt extraction to remove unwanted DNA.
2. Using high concentrations of sodium chloride, approximately 5 M in water or buffer system (physiological pH, for example TRIS or others)
3. Detergent extraction to disrupt cell membranes (lysis) and extract proteins. The detergents used included at least one of Sodium Dodecyl Sulphate (SDS), Triton X-100, Triton X-104, Tween 20, Tween 8, and/or CHAPS.
4. Enzymatic digestion: DNAse and RNAse were use to cleave and remove DNA and RNA from the mammalian tissue at physiological pH. The tissue was contacted with pepsin at low pH.
5. Acid solubilization: Hydrochloric Acid was used at high concentrations and low pH.
6. Intermittent water and/or buffer rinses (preferably at physiological pH and salt concentrations) were performed prior to and/or after each step.

Manufacturing Process Molding—Lyophilization: The final slurry formed by the extraction process may then be processed through lyophilization into a dried semi-solid or solid collagen component. The final configuration of the implant was created generally by the following steps:
1. Final composition containing purified collagen, and other useful components for treating and/or repairing a tissue, and providing desirable structural and dissolution characteristics
2. Gelation
3. Molding
4. Freeze drying
5. Cutting, chopping, grinding, or milling
6. Absorption

Final composition: Once the extraction process was completed, the resulting purified collagen was adjusted to the desired concentration in a range of 10 g/L to 80g/L, 400 mg/g to 10,000 mg/g, or 40 w/w % to 70 w/w %, depending on the application (e.g., torn or ruptured meniscus, ACL, rotator cuff, or combinations thereof). In addition to the purified collagen, one of the following materials was added to the purified collagen:
1. A salt such as calcium, magnesium, other salts, or combinations thereof, which are able to facilitate healing of the ruptured or torn tissue.
2. One or more factors to improve the outcome/healing process post-surgery, for example: growth factors, proteins, e.g., PDGF, VEGF, TGF-β, peptide chains, e.g., CU-GHK, RGD-peptides, proteinase and/or collagenase inhibitors, other molecules know to improve wound healing, e.g., Zn-ions, retinol, vitamins, steroids.
3. Variations of the other components described within this disclosure.

A non-limiting example of a collagen implant composition was prepared having the components shown in Table 1. Those skilled in the art will understand that this example is non-limiting. Other combinations of the described components and/or variations in the composition mixture and amounts in accordance with the entirety of this disclosure are also suitable for implant compositions according to the invention.

TABLE 1
Collagen Implant Composition for forming the Collagen Implants

| | Composition | |
|---|---|---|
| Component | (dry in w/w %) | Range |
| Collagen | 65% | 40 to 70% |
| HEPES | 21% | As needed |
| Calcium | 3% | 2-5% |
| Sodium | 3% | 2-5% |
| Chloride | 7% | 5-11% |
| GAG | <1% | 0-2% |

Gelation: In some instances, a controlled self-assembly of the purified collagen molecules in the implant composition was implemented. The self-assembly process was thermally driven and occurred faster at higher temperatures. The progress of self-assembly was monitored by measuring the viscosity of the material, increasing over time. In this example, the viscosity was equivalent to a soft-boiled egg yolk (e.g., about 0.0181 Pa/s to about 0.0304 Pa/s). To achieve the desired amount of self-assembly, the temperature of the implant composition may be increased from room temperature to about 30° C. to about 37° C. for a period of one hour and up to 8 hours. In this example, the self-assembly step was conducted at 34° C. for a duration of 180 minutes.

Self-assembly and gelation influences the resistance of the implant to resorption in the host and by extension how long the device persists in the body. There is no direct correlation of this, other than even smaller amounts of gelation result in the implant being more stable in the body. Controlled self-assembly and gelation were performed using the following processes:

### Gelation Method A: Pieces With Defined Dimensions

In this example, the implant composition was poured into a metal tray to form a sheet having a thickness of 1 cm to 4 cm. The tray containing the implant composition was then placed in a chamber at 35° C. for 120 min to achieve the desired self-assembly. Afterwards, the tray was transferred into a fridge at 4° C. or lower to cool the material down and slow down the self-assembly process to an essential standstill. The material was then placed in a lyophilizer and freeze dried until achieving a residual moisture level of 3 w/w % to 5 w/w %.

### Gelation Method B: Pieces With Pre-Determined, Defined Dimensions

In this example, the implant composition was poured into a metal tray having a grid received therein to separate the tray into areas of identical size. The grid was formed of a thin metal sheet, similar to an ice cube tray. The tray was configured to form cuboid portions of the implant composition, each having a thickness of 1 cm to 4 cm. The tray containing the implant composition was then placed in a chamber at 35° C. for 120 min to achieve the desired self-assembly. Afterwards, the tray was transferred into a refrigerator at 4° C. or lower to cool the material down and slow down the self-assembly process to an essential standstill. The material was then placed in a lyophilizer and freeze dried until achieving a residual moisture level of 3 w/w % to 5 w/w %.
Gelation Example B.1: In one configuration, the grid was part of the tray, such that the individual segments were filled with a defined volume of the implant composition to achieve a final amount of material of each cuboid to be 2.0 g.
Gelation Example B.2: In another configuration, the grid was a separate component and inserted into the tray after the material was poured into it. That way the material was separated into equal weighted aliquots by the metal sheets making up the grid.

Thermal re-assembly (Gelation) may be further employed to achieve self-assembly of the solubilized collagen to control the ability and speed of the implant to form a suspension with blood. No gelation results in fast blood absorption. Gelation for 180 min at 34° C. resulted in blood absorption over approximately 4 min. The resorption profile of the implant during the healing process also may be impacted by gelation. Gelation for 150 min at 35° C. results in an absorption time of approximately 35 to 42 days.

The trays were then placed in a chamber at 32° C. for 240 mins to achieve the desired amount of self-assembly. Afterwards, the tray was transferred into a refrigerator at 4° C. or lower to cool the material down and slow down the self-assembly process to an essential standstill. The material was then placed in a lyophilizer and freeze dried to achieve a residual moisture level of less than 2 w/w %.

There are many possible variations of the lyophilization tray that can be implemented. For example, the tray and/or grid can form a three-dimensional shape based on any geometry such as a square, rectangle, circle, oval, hexagon, rhomboid or may even be irregularly shaped. The floor of the tray may be an integral part of the tray or may be separatable from the outer frame and/or the inner grid if it exists. The inner grid can be made of a single piece with a fixed, defined size of the compartments therein or made up to form individual bars that allow for multiple varied sizes of compartments within the tray. The inner grid may be configured such that it does not extend all the way through the material from the bottom of the tray, but only partially, for example 30% to 50% of the depth. In this case, the sheets can be broken into pieces of a desired size after lyophilization, similar to chocolate bars.

Final shaping of the implant: Several variations of a process for shaping an injectable collagen implant were investigated. The size of the semi-solid or solid component, particles, pieces etc. was varied in consideration of injecting the implant from a variety of syringes and cannula sizes after absorption with the patient's own blood.

### Removal Process

The configuration of the lyophilization tray drives the removal process of the dried material from within it. In one embodiment, the tray was turned upside down and either the sheet or the portions of a defined size were released.

In another method, the dried implants each having a defined size were pushed or pulled out of the mold either manually or by a tool. Alternatively the tray or the individual wells were coated with a removable lining that released the sheet or the defined portions when removed.

The trays, if made from a flexible material (e.g., silicone), can be pulled onto a drum with the opening of the tray or wells facing outwards to release the sheet or the individual portions by expanding the material of the tray/walls of each well so that gravitational forces are sufficient to remove them.

### Portioning of the Material to the Desired Amount

This optional step was advantageous after forming lyophilized sheets that could be subsequently cut or divided to form a plurality of pieces or particles having a desired mean size. In the case of a tray having individual wells that lyophilize the material in predetermined amounts, this step may be omitted because the nature of the tray and its wells ensures the correct amount for each piece. If the material is lyophilized as a single sheet, then the material may first be separated into smaller pieces, for example, by cutting with a blade/scissors, a bandsaw-like device, a die cutter, or similar laboratory equipment. The die cutter has the advantage that it would directly create pieces of the final desired size (portion).

In case of the tray having compartments whose walls do not fully partition the material, but form areas of thinner material thickness, thereby forming pre-determined break lines, similar to a bar of chocolate, the material is broken apart. The breaking occurs at the areas of thinner material and can be facilitated by breaking over an edge/blade or a corner, or cutting with a blade or similar device. In one example, the breaking occurs first in one direction than in another, following the areas of thinner material. The final pieces may be of a defined range or size, geometry and weight.

In an alternate tray design, there are compartments, but parts of the walls extend all the way through the material creating areas along the walls of the compartment where there is no material and areas where the material is less in thickness than in the bulk of the material. This would look similar to a bar of chocolate with perforations between the pieces. In subsequent steps, the material may be broken into pieces of defined weight, size and geometry. In the example of a tray with separated compartments, this step can be omitted if the compartments are of a size that matches the final desired size, for example, a final desired particle size of greater than 1 mm (in any dimension) to 100 mm (in any dimension). If the step is performed, it may be performed with partitioned materials to control the final amount. The material may be cut, sliced, grated, shredded, ground, or milled, or any combination thereof. If a defined particle size or particle size range is desired, size exclusion separation techniques may be employed. If this is not required, the material may be used without size exclusion techniques.

It was determined that the portioned implants could be fed into a cylinder having one or more rotating blades therein. Depending on the speed of the blades and the rate of feeding the material, the resulting particles may vary in size. Boths parameters can be used to control the particle size. The material may alternatively be fed into a container with rotating blades at the bottom. Cutting would be controlled similar to food processing by time, pulse, speed, and/or blade design. The cutting and size exclusion separation may be combined by using a rotating blade that is fundamentally round and has integrated blades. Such blades are configured to continually shave off material from the larger piece of material with the material falling down through the blade similar to food grinders or shredders. The cut size may be controlled by the rise of the cutting blade above the level of the of the plane of the blade's rotation and can vary widely (up to 10 mm or more), and by the shape of the blade (straight, concave, convex, angled, v or u-shaped) relative to the plane of the blade's rotation.

### Cutting Example A (Continued From Gelation)

Cutting: The resulting collagen sheet was removed from the tray and then cut into pieces of a certain size to yield the required mass for the device, for example 2.0 g. The exact mass is determined by the desired application and can be anywhere from a few micrograms up to several hundreds of grams. The smaller, uniformly weighted pieces, representing the final weight of the device were then cut with a blade or a band saw into smaller pieces of approximately 3 mm×3 mm×3 mm. Notably, the size of these pieces may be varied depending on the requirements of the application. All of the pieces were then transferred into a glass vial, which was scaled and then sterilized.

### Cutting Example B (Continued From Gelation)

No further sizing: In both examples the dry material is then removed from its compartments, yielding pieces of a desired and pre-determined size for the intended application, for example 5 mm×5 mm×5 mm. A defined number of these pieces, for example 15 to yield a final weight 1.8 g are then filled into a plastic vial, sealed and sterilized.

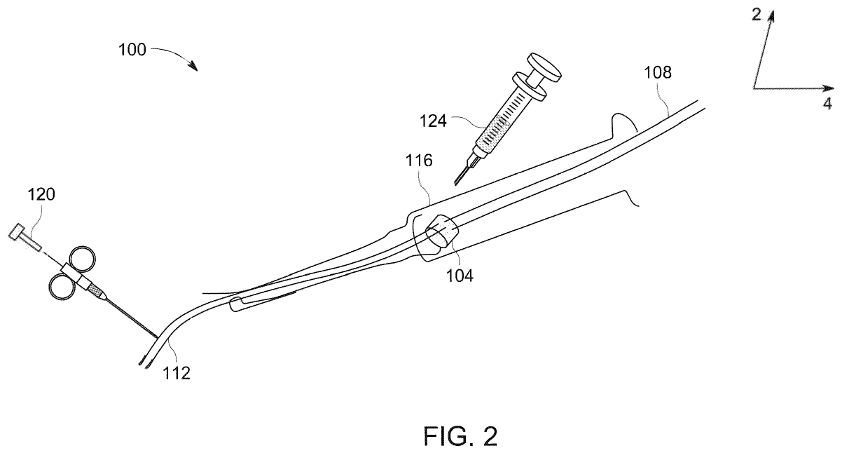

Grating: Cut sheet into strips, then feed strips into a hopper with rotating bladed at the bottom, cutting the strips into slices (see FIG. 4 for examples). Thickness of slices is defined by the speed of the blade and the advancing speed of the strip into the path of the blade (similar to extrusion processes). This can yield very thin slices (shavings) or thicker slices. Generally the thickness of the slices is less than their circumference.

Blending/shredding: Implant sheets formed by lyophilization may be cut into smaller pieces (e.g., undefined size, defined size) and then placed into a container with rotating blades in the bottom, similar to a food processor. To control the resulting particle size, the following parameters may be varied: blade design, length, width, geometry (straight, bent, wavy), cross-section, blade speed (e.g., from 10 revs/s to 50,000 revs/s), blending duration (e.g., continuous from 0.5 s to 15 s or more), pulsed with similar durations as above. The container may be cooled to prevent any heat damage to the material form the exposure to the process.

Grinding/Milling: The implants may be pre-cut into smaller pieces of an undefined size and then fed in batches into a mill to grind the material down to form a powder. Any type of mill is suitable (e.g., a ball mill or a hammer mill). Using a ball mill, the following parameters may be controlled to achieve the desired result: number of balls in chamber, size of balls in the chamber (notably, the balls do not need to be of a single size), materials of balls in the chamber (note the balls do not all need to be of the same material), ratio of ball size/number to chamber size, speed of rotation of chamber, and/or the chamber might need to be cooled to approximately 4° C. to avoid thermal material damage. Using a hammer mill, the following parameters may need to be controlled to achieve the desired result: hammer speed, amount of material filled into the chamber, distance of hammer from the frame, and/or the chamber may need to be cooled (e.g., to 4° C.) to prevent heat damage to the material. In order to keep the material cool and to increase its brittleness, dry ice or liquid nitrogen was added to the chamber. Both were removed subsequently via simple evaporation.

Size Exclusion Separation (SES): After the milling, cutting, grating, grinding, etc. process, the resulting particles were separated into a desired particle size, depending on the application. In an extreme case, the application may have no requirements for a desired particle size and no size exclusion separation technique may be needed. Four (4) SES methods were evaluated: to achieve a mix of particles with a defined minimum size (low cut off), to achieve a mix of particles with a defined maximum size (high cut off), to achieve a mix of particles with a defined minimum and maximum size (range), and/or to combine more than one size range of particles, if that is required (e.g., a combination of particles with a range of 50 μm to 400 μm and 800 μm to 900 μm, a combination of particles no bigger than 250 μm and no smaller than 500 μm), or combinations thereof. Various forms of sieving were investigated:

Dry sieving: Dry sieving is commonly used for particle size analysis and separation. It involved passing the material through the sieve with openings of varied sizes. The finer particles passed through the sieve, while larger particles were retained. This method was highly efficient and widely employed due to its simplicity and reliability.

Vibratory sieving: Vibratory sieves utilized vibrations to enhance the sieving process. The sieve was subjected to oscillatory movements, causing the material to move rapidly, facilitating separation. Vibratory sieving was highly effective in achieving precise and efficient separation, particularly for fine powders and granules. Its ability to minimize blinding and improve throughput makes it a popular choice in industries such as pharmaceuticals and chemical processing.

Centrifugal Sieving: Centrifugal sieving involved the use of centrifugal force to enhance the separation process. The sieve was rotated at high speeds, creating a centrifugal acceleration that aids in the separation of particles based on size and density. This technique was advantageous for achieving high throughput and accurate separation when fine particles and powdered materials were needed.

Air Jet Sieving: Air jet sieving employed a stream of air to fluidize the material sieved, allowing the finer particles to pass through the sieve. Choosing stainless steel sieve screens for particle analysis helped the air jet effectively disperse and separate the particles based on their. size, resulting in precise classification. This technique was commonly used in industries where high accuracy and repeatability are crucial, such as the pharmaceutical and food industries.

Tapping Sieving: Tapping sieving utilized mechanical tapping or striking action to assist in the sieving process. Operators subjected the sieve to tapping or knocking, which assisted in breaking down agglomcrates and improving separation efficiency. Industries, such as construction, mining, and pharmaceuticals, widely utilize this technique for particle size analysis. This method was well suited to achieve a defined particle size distribution by arranging a series of sieves with different cut-offs in sequence (stacked on top of each other).

Rotary Sieving: Rotary sieving involved the use of a rotating sieve drum to separate materials based on size. Operators fed the material into the drum, and as it rotated, the sieve openings allowed smaller particles to pass through while retaining larger particles. Rotary sieves are versatile and widely employed in various industries for efficient particle separation.

Multilayer Sieving: Multilayer sieving involved using multiple layers of sieves stacked on top of each other to achieve more precise separation. Each sieve layer had different openings, allowing for finer classification of materials. Industries, such as pharmaceuticals and fine chemicals, employ multilayer sieving when high precision and accurate separation are of utmost importance.

Combination of milling and SES: The following process created smaller particles from the lyophilized sheet and by combining milling and sieving methods to achieve the desired particle size in a single processing step. A hammer mill was used having a limited hammer speed, while the system was cooled to prevent heat damage during milling. An implant sheet was fed into a blender to create smaller pieces, then the smaller pieces were transferred into a grinder to get them to a desired particle size, for example, 500 μm to 2,000 μm. The sheet was fed into a blender to create smaller pieces, then the smaller pieces were transferred into a mill to grind them to a desired particle size, for example 50 μm to 120 μm, or 10 to 50 μm.

### Further Variations to the Implant Compositions to Achieve Desired Properties

Viscosity: The final properties of the implant composition and/or implant (e.g., semi-solid, solid, injectable) may be determined by the final viscosity. The collagen implant may be divided into particles, flakes, chunks, etc. and suspended or gelled in blood (itself a suspension) having a desired viscosity. The manufacturing process controls the solid element of the final suspension being applied, mostly in terms of size and shape. The amount of blood used is defined by the requirements of the surgical procedure, mostly in terms of volume. Other exemplary considerations include blood coagulation, heparinized blood (slowest), heparinized blood with calcium ions added (slow), whole blood (faster), whole blood with Calcium added (fastest). As a result viscosity can be varied broadly by changing one or more of these parameters.

Porosity: The porosity of the collagen particles is related to the amount of fluid (e.g., blood) that they can absorb. The bigger the particles the more important porosity of the particles becomes for application. Blood absorption into collagen is suitable to turn the material into a suspension, making the solids malleable (gel-like) and suitable to push the suspension through a syringe. Such suspensions may allow larger particles to go through a narrower opening. Implant materials evaluated herein had a porosity of about 80% to about 99%. Generally, the smaller the particles, the less important the porosity becomes. For particles smaller than 1 mm, a porosity of 10% or less may be possible.

### Applicator

The applicator employed for ejection of the implant, had a tubular structure of sufficient length to reach the desired anatomical site, and sufficient diameter to apply the blood saturated implant with an ample volume and viscosity to fill the desired space. The means of advancing/ejecting the collagen implant was a longitudinal piece that was inserted into the tubular structure from one end and by advancing it along the length of the tubular structure displacing the collagen implant out of the other end of the tubular structure. Notably, the tubular structure can be of any cross section and the inner and outer diameter can be consistent or variable along its length (e.g., the diameter of the tubular structure can narrow towards the exits to allow for better control of the placement of the collagen implant). The longitudinal piece may be solid and of a constant cross-section or it could have a variable cross-section. The material of construction may be suitable to allow for adjustment of its diameter to the inner diameter and geometry of the tubular structure. In this example, the applicator was a syringe. During the surgical procedure, the collagen implant was placed in the barrel of the syringe and then a defined amount of the patient's own blood was added to the barrel of the syringe. The barrel had a marking to indicate the volume of blood required (range, min, maximum amount or combinations thereof). Notably, the applicator can include, for example as part of the piston, a means (e.g., a propeller, baffles, internal mixer, etc.) to mix the collagen implant and the autologous blood in the barrel of the syringe.

### Example 2

### Surgical Repair of a Tissue Using a Collagen Implant

In this example, the collagen implant was comprised of particles have a mean size of about 500 μm to about 2,000 μm with a total mass of 1.8 g. The applicator was a syringe with a volume of 20 ml, the barrel of the syringe and the piston being separated. The collagen implant was filled into the barrel of the syringe, which in turn was sealed at the top and the bottom.

The filled barrel of the syringe and its matching piston were placed in a tray, designed to physically separate the two components and keep them stable in place by means of two deep drawn cavities in a plastic blister. The blister was sealed with an air-and moisture barrier, that is, a standard foil material used in medical packaging. The blister was placed in a carton which was sufficiently strong to protect the product during transit from any damage or loss of seal integrity.

During surgery, the surgeon removed the seal from the back end of the syringe and filled between 4 ml and 7 ml of the patient's own blood into the barrel of the syringe. Subsequently, the piston was inserted into the barrel. Shaking the applicator achieved the desired mixing of the blood and the collagen implant to prepare the implant for use.

To place the implant, the seal at the front of the syringe was removed. If required, a cannula was attached to the front of the syringe to facilitate the delivery of the, now blood saturated collagen implant, into difficult to reach anatomical spaces. The implant was placed by depressing the plunger and ejecting the implant (through the cannula) into the desired anatomical location. The size of the cannula was chosen to facilitate ejection of the implant. In this example, the collagen implant was in the form of compressible particles absorbed with the patient's blood and suitable for ejection through cannula having a diameter of greater than about 2,000 μm. The collagen implant also could have been in the form of a single semi-solid or solid piece that is compressible and deformable, configured for ejection through a suitable sized cannula.

### Example 3

### Surgical Repair of a Tissue Using a Collagen Implant

In this example, the collagen implant was comprised of particles with a mean size of less than 2,500 μm and a total mass of 1.5 g. The collagen implant was filled into a glass vial with a hermetic seal and a narrow neck opening to facilitate the process of removing the implant form the vial and into the applicator without a funnel.

The applicator was an open bore syringe with a volume of 25 ml. The open bore at the lower end was sealed by a screwcap.

Both components were placed in in a folded paper tray with two compartments to keep the vial and the applicator separate. The paper tray was equipped with a lid (similar to a regular gift box or folding box) and protected the components during transit.

During surgical use, the applicator was prepared by removing the piston from the syringe and putting it aside. Then the vial was opened and the collagen implant was directly filled into the barrel of the syringe. Subsequently, 8 ml of autologous blood were filled into the barrel of the syringe and the piston was inserted back into the barrel. Shaking the applicator achieved the desired mixing of the blood and the collagen implant to prepare the implant for use.

To place the implant, the screwcap at the front of the applicator was removed, the applicator was then ready for use. The surgeon inserted the applicator and guided it to the desired anatomical space and then injected the implant by depressing the plunger. The size of the cannula was chosen to facilitate ejection of the implant. In this example, the collagen implant was in the form of compressible particles absorbed with the patient's blood and suitable for ejection through cannula having a diameter of greater than about 2,500 μm. The collagen implant also could have been in the form of a single semi-solid or solid piece that is compressible and deformable, configured for ejection through a suitable sized cannula.

In the present disclosure, a subject includes, but is not limited to, any mammal, such as human, non-human primate, mouse, rat, dog, cat, horse or cow. In certain embodiments, a subject is a human. The present disclosure may also include kits for repair of ruptured or torn meniscus and cartilage. A kit may include an implant of the invention having at least one delivery device that carries that collagen implant and instructions for use. The implant may further include one or more sutures that attach a fixation device to the implant. A kit may further include a container that contains a repair material as described herein.

The foregoing written specification is considered to be sufficient to enable one skilled in the art to practice the invention. The present disclosure is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention. Those skilled in the art will recognize or be able to ascertain using no more than routine experimentation, many equivalents to the specific embodiments of the invention described herein. Such equivalents are intended to be encompassed by the following claims.

What is claimed is:

1. A tissue repair system for a tear of a meniscus, comprising:
a collagen implant having a lower surface, an upper surface opposite the lower surface, and a side wall that extends from the lower surface to the upper surface, the side wall defining an outer perimeter that is shaped to fit between a femur and a tibia and along the tear of the meniscus when positioned adjacent to the meniscus, the collagen implant having a self-assembly of collagen fibers and being substantially free of cross-linking agents, wherein the collagen implant is configured for securement to a first and second portion of a meniscus in order to facilitate growth of meniscal tissue at or near the tear.

2. The tissue repair system of claim 1, further comprising one or more suture assemblies configured to pass through one or more of a first portion of the meniscus and a second portion of the meniscus to secure the collagen implant in place on or along the tear of the meniscus.

3. The tissue repair system of claim 1 or 2, further comprising a delivery device configured to carry the collagen implant for delivery at the tear of the meniscus, the delivery device having an elongated body, a proximal end, a distal end opposite the proximal end, and a channel that extends from the distal end toward the proximal end, the channel defining a cross-sectional dimension that is shaped and sized to carry the collagen implant.

4. The tissue repair system of claim 2 or 3, wherein the one or more suture assemblies include:
a first suture assembly configured to pass through the collagen implant and a first portion of the meniscus; and
a second suture assembly configured to pass through the collagen implant and one or more of the first portion of the meniscus and a second portion of the meniscus.

5. The tissue repair system of claim 4, further comprising a suture passer configured to carry and pass: 1) the first suture assembly through the first portion of the meniscus and the collagen implant, and 2) the second suture assembly through the second portion of the meniscus and the collagen implant.

6. The tissue repair system of any preceding claim, further comprising a syringe configured to hold a fluid and inject the fluid into the collagen implant.

7. The tissue repair system of claim 6, wherein the fluid is blood.

8. The tissue repair system of any one of claims 2 to 7, wherein the collagen implant and the one or more suture assemblies are preloaded into the delivery device.

9. The tissue repair system of any one of claims 2 to 8, wherein the collagen implant and the one or more suture assemblies are provisioned outside of the delivery device but are insertable through the delivery device.

10. The tissue repair system of any preceding claim, wherein the collagen implant further comprises a repair material.

11. The tissue repair system of claim 10, wherein the repair material is a platelet, plasma, or blood.

12. The tissue repair system of any preceding claim, wherein the collagen implant comprises at least one of:
collagen in an amount of about 40 w/w % to about 70 w/w %,
at least one salt in an amount of about 2 w/w % to about 5 w/w %,
at least one electrolyte in an amount of about 5 w/w % to about 11 w/w %, or
glycosaminoglycan in an amount of less than about 2 w/w %, or about 0.001 w/w % to less than about 2 w/w %.

13. The tissue repair system of any preceding claim, wherein the collagen implant comprises at least one of:
a collagen content of greater than 400 mg/g, or about 400 mg/g to about 10,000 mg/g,
a GAG content of greater than 100 μg/g, or greater than about 100 μg/g to about 8,000 μg/g,
a DNA content of less than 50,000 ng/g, or about 5 ng/g to less than about 50,000 ng/g,
a phospholipid count of less than 3,000 μM/g, or about 1 μM/g to less than about 3,000 μM/g, or
a pepsin content of less than 12.5 mg/g, or about 0.1 mg/g to less than about 12.5 mg/g.

14. The tissue repair system of any preceding claim, wherein the collagen implant further comprises a buffer.

15. The tissue repair system of claim 12 or 13, wherein the salt comprises calcium, magnesium, or combinations thereof.

16. The tissue repair system of claim 12 or 13, wherein the electrolyte comprises chloride, nitrate, sulfate, silicate, phosphate, aluminate, sodium tetraborate, or combinations thereof.

17. The tissue repair system of claim 12 or 13, wherein the electrolyte comprises chloride.

18. The tissue repair system of any preceding claim, wherein the collagen implant comprises a plurality of particles that are injectable toward a tissue site.

19. The tissue repair system of claim 18, wherein the plurality of particles is suspended in blood.

20. The tissue repair system of any preceding claim, wherein the collagen implant comprises a porosity of at least about 80%, or about 80% to 99%.

21. A tissue repair system for a tear of a meniscus, comprising:
a collagen implant having a lower surface, an upper surface opposite the lower surface, and a side wall that extends from the lower surface to the upper surface, the side wall defining an outer perimeter that is shaped to fit between a femur and a tibia and along the tear of the meniscus when positioned adjacent to the meniscus, the collagen implant being substantially free of cross-linking agents, wherein the collagen implant is configured for securement to a first and second portion of a meniscus in order to facilitate growth of meniscal tissue at or near the tear;
one or more suture assemblies configured to pass through one or more of a first portion of the meniscus and a second portion of the meniscus to secure the collagen implant in place on or along the tear of the meniscus; and
a delivery device configured to carry the collagen implant for delivery at the tear of the meniscus, the delivery device having an elongated body, a proximal end, a distal end opposite the proximal end, and a channel that extends from the distal end toward the proximal end, the channel defining a cross-sectional dimension that is shaped and sized to carry the collagen implant.

22. The tissue repair system of claim 21, wherein the collagen implant comprises at least one of:
collagen in an amount of about 40 w/w % to about 70 w/w %,
at least one salt in an amount of about 2 w/w % to about 5 w/w %,
at least one electrolyte in an amount of about 5 w/w % to about 11 w/w %, or
glycosaminoglycan in an amount of less than about 2 w/w %, or about 0.001 w/w % to less than about 2 w/w %.

23. The tissue repair system of claim 21 or claim 22, wherein the collagen implant comprises at least one of:
a collagen content of greater than 400 mg/g, or about 400 mg/g to about 10,000 mg/g,
a GAG content of greater than 100 μg/g, or greater than about 100 μg/g to about 8,000 μg/g,
a DNA content of less than 50,000 ng/g, or about 5 ng/g to less than about 50,000 ng/g,
a phospholipid count of less than 3,000 μM/g, or about 1 μM/g to less than about 3,000 μM/g, or
a pepsin content of less than 12.5 mg/g, or about 0.1 mg/g to less than about 12.5 mg/g.

24. The tissue repair system of claim 22 or claim 23, wherein the collagen implant further comprises a buffer.

25. The tissue repair system of any one of claims 21 to 24, wherein the collagen implant comprises a porosity of at least about 80%, or about 80% to 99%.

26. The tissue repair system of any one of claims 21 to 25, wherein the collagen implant is injectable from the delivery device.

27. A method, comprising:
forming a portal in a knee joint to access a meniscus having a rupture or tear;
inserting a delivery device into the portal so that a distal end of the delivery device is positioned close to the meniscus;
causing a collagen implant to exit the distal end of the delivery device in order to position it on or near the rupture or tear of the meniscus; and
securing, with one or more suture assemblies, the collagen implant in place at or near the rupture or tear of the meniscus,
wherein the collagen implant is a self-assembly of collagen fibers, is substantially free of cross-linking agents, and is configured to facilitate growth of meniscal tissue at or near the tear.

28. The method of claim 27, further comprising injecting a volume of blood into the collagen implant.

29. The method of claim 27 or 28, further comprising injecting a volume of blood into the collagen implant before the collagen implant exits the distal end of the delivery device.

30. The method of any one of claims 27 to 29, wherein securing, with one or more suture assemblies the collagen implant, in place at the rupture or tear of the meniscus includes:
threading a first suture assembly onto a first portion of the meniscus; and
threading a second suture assembly onto a second portion of the meniscus.

31. The method of claim 30, further comprising threading the collagen implant along or on at least one of the first suture assembly and the second suture assembly.

32. The method of any one of claims 27 to 31, wherein causing the collagen implant to exit the distal end of the delivery device comprises injecting the collagen implant at or near the rupture or tear of the meniscus.

33. The method of any one of claims 27 to 32, wherein the collagen implant comprises at least one of:
collagen in an amount of about 40 w/w % to about 70 w/w %,
at least one salt in an amount of about 2 w/w % to about 5 w/w %,
at least one electrolyte in an amount of about 5 w/w % to about 11 w/w %, or
glycosaminoglycan in an amount of less than about 2 w/w %, or about 0.001 w/w % to less than about 2 w/w %.

34. The method of any one of claims 27 to 33, wherein the collagen implant comprises at least one of:
a collagen content of greater than 400 mg/g, or about 400 mg/g to about 10,000 mg/g,
a GAG content of greater than 100 μg/g, or greater than about 100 μg/g to about 8,000 μg/g,
a DNA content of less than 50,000 ng/g, or about 5 ng/g to less than about 50,000 ng/g,
a phospholipid count of less than 3,000 μM/g, or about 1 μM/g to less than about 3,000 μM/g, or
a pepsin content of less than 12.5 mg/g, or about 0.1 mg/g to less than about 12.5 mg/g.

35. A method, comprising:
positioning a bone fixation device on a femur, wherein one or more suture assemblies are attached to the bone fixation device;
securing the one or more suture assemblies to an anterior cruciate ligament (ACL) with a rupture or tear;
threading a collagen implant along or on the one or more suture assemblies;
injecting a volume of blood into the collagen implant so that the blood is positioned at or adjacent the rupture or tear of the ACL and a rupture or tear of a meniscus; and
observing growth of ACL and meniscal tissue at or near a rupture or tear of the ACL and the meniscus.

36. The method of claim 35, further comprising coupling the collagen implant to the femur with the bone fixation device.

37. The method of claim 35 or 36, wherein the collagen implant is substantially free of cross-linking agents, and is configured to facilitate growth of meniscal tissue at or near the tear.

38. The method of any one of claims 35 to 37, wherein the collagen implant comprises at least one of:
collagen in an amount of about 40 w/w % to about 70 w/w %,
at least one salt in an amount of about 2 w/w % to about 5 w/w %,
at least one electrolyte in an amount of about 5 w/w % to about 11 w/w %, or
glycosaminoglycan in an amount of less than about 2 w/w %, or about 0.001 w/w % to less than about 2 w/w %.

39. The method of any one of claims 35 to 38, wherein the collagen implant comprises at least one of:
a collagen content of greater than 400 mg/g, or about 400 mg/g to about 10,000 mg/g,
a GAG content of greater than 100 μg/g, or greater than about 100 μg/g to about 8,000 μg/g,
a DNA content of less than 50,000 ng/g, or about 5 ng/g to less than about 50,000 ng/g,
a phospholipid count of less than 3,000 μM/g, or about 1 μM/g to less than about 3,000 μM/g, or a pepsin content of less than 12.5 mg/g, or about 0.1 mg/g to less than about 12.5 mg/g.

40. The method of claim 35, claim 38 or claim 39, wherein the collagen implant further comprises a buffer.

41. The method of any one of claims 35 to 40, wherein the collagen implant comprises a porosity of at least about 80%, or about 80% to 99%.

42. A method, comprising:
positioning a bone fixation device on a femur, wherein one or more suture assemblies are attached to the bone fixation device;
securing the one or more suture assemblies to an anterior cruciate ligament (ACL) with a rupture or tear;
injecting a volume of blood into a collagen implant;
threading the collagen implant along or on the one or more suture assemblies so that the blood is positioned at or adjacent the rupture or tear of the ACL and a rupture or tear of a meniscus; and
observing growth of ACL and meniscal tissue at or near a rupture or tear of the ACL and a meniscus.

43. The method of claim 42, further comprising coupling the collagen implant to the femur with the bone fixation device.

44. The method of claim 42 or 43, wherein the collagen implant is substantially free of cross-linking agents, and is configured to facilitate growth of meniscal tissue at or near the tear.

45. The method of any one of claims 42 to 44, wherein the collagen implant comprises at least one of:
collagen in an amount of about 40 w/w % to about 70 w/w %,
at least one salt in an amount of about 2 w/w % to about 5 w/w %,
at least one electrolyte in an amount of about 5 w/w % to about 11 w/w %, or
glycosaminoglycan in an amount of less than about 2 w/w %, or about 0.001 w/w % to less than about 2 w/w %.

46. The method of any one of claims 42 to 45, wherein the collagen implant comprises at least one of:
a collagen content of greater than 400 mg/g, or about 400 mg/g to about 10,000 mg/g,
a GAG content of greater than 100 μg/g, or greater than about 100 μg/g to about 8,000 μg/g,
a DNA content of less than 50,000 ng/g, or about 5 ng/g to less than about 50,000 ng/g,
a phospholipid count of less than 3,000 μM/g, or about 1 μM/g to less than about 3,000 μM/g, or
a pepsin content of less than 12.5 mg/g, or about 0.1 mg/g to less than about 12.5 mg/g.

47. The method of any one of claims 42 to 46, wherein the collagen implant further comprises a buffer.

48. The method of any one of claims 42 to 46, wherein the collagen implant comprises a porosity of at least about 80%, or about 80% to 99%.

49. A tissue repair system for a tear of a rotator cuff, comprising:
a collagen implant having a lower surface, an upper surface opposite the lower surface, and a side wall that extends from the lower surface to the upper surface, the side wall defining an outer perimeter that is shaped to fit in a shoulder joint along the tear on the rotator cuff, the collagen implant including a self-assembly of collagen fibers and being substantially free of cross-linking agents, wherein the collagen implant is configured for securement to a first and second portion of a tissue on a rotator cuff in order to facilitate growth of tissue at or near the tear of the rotator cuff.

50. The tissue repair system of claim 49, further comprising one or more suture assemblies configured to pass through one or more of a first portion of the rotator cuff and a second portion of the rotator cuff to secure the collagen implant in place on or along the tear of the rotator cuff.

51. The tissue repair system of claim 49 or 50, wherein the one or more suture assemblies include:
a first suture assembly configured to pass through the collagen implant and a first portion of the rotator cuff; and
a second suture assembly configured to pass through the collagen implant and one or more of the first portion of the rotator cuff and a second portion of the rotator cuff.

52. The tissue repair system of any one of claims 49 to 51, further comprising a delivery device configured to carry the collagen implant in a compressed state for delivery at or near the tear of the rotator cuff, the delivery device having an elongated body, a proximal end, a distal end opposite the proximal end, and a channel that extends from the distal end toward the proximal end, the channel defining a cross-sectional dimension that is shaped and sized to carry the collagen implant.

53. The tissue repair system of claim 52, wherein the delivery device includes a moveable rod in the channel and is configured to eject the collagen implant from the distal end of the delivery device into tissue.

54. The tissue repair system of claim 52, wherein the collagen implant and the one or more suture assemblies are preloaded into the delivery device.

55. The tissue repair system of claim 52, wherein the collagen implant and the one or more suture assemblies are provisioned outside of the delivery device but are insertable through the delivery device.

56. The tissue repair system of any one of claims 49 to 55, wherein the collagen implant further comprises a repair material.

57. The tissue repair system of claim 56, wherein the repair material is a platelet, plasma, or blood.

58. The tissue repair system of any one of claims 49 to 57, wherein the collagen implant comprises at least one of:
collagen in an amount of about 40 w/w % to about 70 w/w %,
at least one salt in an amount of about 2 w/w % to about 5 w/w %,
at least one electrolyte in an amount of about 5 w/w % to about 11 w/w %, or
glycosaminoglycan in an amount of less than about 2 w/w %, or about 0.001 w/w % to less than about 2 w/w %.

59. The tissue repair system of any one of claims 49 to 58, wherein the collagen implant comprises at least one of:
a collagen content of greater than 400 mg/g, or about 400 mg/g to about 10,000 mg/g,
a GAG content of greater than 100 μg/g, or greater than about 100 μg/g to about 8,000 μg/g,
a DNA content of less than 50,000 ng/g, or about 5 ng/g to less than about 50,000 ng/g,
a phospholipid count of less than 3,000 μM/g, or about 1 μM/g to less than about 3,000μ M/g, or
a pepsin content of less than 12.5 mg/g, or about 0.1 mg/g to less than about 12.5 mg/g.

60. The tissue repair system of any one of claims 49 to 59, wherein the collagen implant further comprises a buffer.

61. The tissue repair system of claim 58 or claim 59, wherein the salt comprises calcium, magnesium, or combinations thereof.

62. The tissue repair system of claim 58 or claim 59, wherein the electrolyte comprises chloride, nitrate, sulfate, silicate, phosphate, aluminate, sodium tetraborate, or combinations thereof.

63. The tissue repair system of any one of claims 49 to 62, wherein the collagen implant comprises a plurality of particles that are injectable toward a tissue site.

64. The tissue repair system of claim 63, wherein the plurality of particles is suspended in blood.

65. The tissue repair system of any one of claims 49 to 64, wherein the collagen implant comprises a porosity of at least about 80%, or about 80% to 99%.

66. A method, comprising:
attaching a first fixation device to a humerus;
attaching a second fixation device to a rotator cuff tendon;
connecting one or more suture assemblies to the first and second fixation devices;
placing a collagen implant injected with blood along or the one or more suture assemblies; and
positioning the collagen implant between ruptured or torn ends of the rotator cuff tendon.

67. The method of claim 66, further comprising injecting blood into the collagen implant.

68. The method of claim 66 or 67, further comprising threading the collagen implant along or on a the one or more suture assemblies.

69. The method of any one of claims 66 to 68, wherein the collagen implant comprises at least one of:
collagen in an amount of about 40 w/w % to about 70 w/w %,
at least one salt in an amount of about 2 w/w % to about 5 w/w %,
at least one electrolyte in an amount of about 5 w/w % to about 11 w/w %, or
glycosaminoglycan in an amount of less than about 2 w/w %, or about 0.001 w/w % to less than about 2 w/w %.

70. The method system of any one of claims 66 to 69, wherein the collagen implant comprises at least one of:
a collagen content of greater than 400 mg/g, or about 400 mg/g to about 10,000 mg/g,
a GAG content of greater than 100 μg/g, or greater than about 100 μg/g to about 8,000 μg/g,
a DNA content of less than 50,000 ng/g, or about 5 ng/g to less than about 50,000 ng/g,
a phospholipid count of less than 3,000 μM/g, or about 1 μM/g to less than about 3,000 μM/g, or
a pepsin content of less than 12.5 mg/g, or about 0.1 mg/g to less than about 12.5 mg/g.

71. The method of any one of claims 66 to 70, wherein the collagen implant further comprises a buffer.

72. The method of claim 69 or claim 70, wherein the salt comprises calcium, magnesium, or combinations thereof.

73. The method of claim 69 or claim 70, wherein the electrolyte comprises chloride, nitrate, sulfate, silicate, phosphate, aluminate, sodium tetraborate, or combinations thereof.

74. The method of claim 73, wherein the electrolyte comprises chloride.

75. The method of any one of claims 66 to 74, wherein the collagen implant comprises a plurality of particles that are injectable toward a tissue site.

76. The method of claim 75, wherein the plurality of particles is suspended in blood.

77. The method of any one of claims 66 to 77, wherein the collagen implant comprises a porosity of at least about 80%, or about 80% to 99%.
